SEQUENCE LISTING (89 sequences)

SEQ ID NO: 1 - protein, 457 aa, Artificial sequence
  Synthetic peptide
METDTLLLWVLLLWVPGSTGEIVLTQSPATLSLSPGERATLSCRASQSVSSYLAWYQQKPGQAPRLLIYDASNRATGIPARFSGSGSGTDFTLTISSLEPEDFAVYYCQQRSNWLMYTFGQGTKLEIKGSTSGSGKPGSGEGSTKGQVQLVQSGSELKKPGASVKISCKASGYTFTSYAMNWVRQAPGQGLESMGWINTNTGNPTYAQGFTGRFVFSMDTSVSTAYLQISSLKAEDTAIYYCAPRYSSSWYLDYWGQGTLVTVSSESKYGPPCPPCPFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 564 aa, Artificial sequence
  Synthetic peptide
METDTLLLWVLLLWVPGSTGEIVLTQSPATLSLSPGERATLSCRASQSVSSYLAWYQQKPGQAPRLLIYDASNRATGIPARFSGSGSGTDFTLTISSLEPEDFAVYYCQQRSNWLMYTFGQGTKLEIKGSTSGSGKPGSGEGSTKGQVQLVQSGSELKKPGASVKISCKASGYTFTSYAMNWVRQAPGQGLESMGWINTNTGNPTYAQGFTGRFVFSMDTSVSTAYLQISSLKAEDTAIYYCAPRYSSSWYLDYWGQGTLVTVSSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 674 aa, Artificial sequence
  Synthetic peptide
METDTLLLWVLLLWVPGSTGEIVLTQSPATLSLSPGERATLSCRASQSVSSYLAWYQQKPGQAPRLLIYDASNRATGIPARFSGSGSGTDFTLTISSLEPEDFAVYYCQQRSNWLMYTFGQGTKLEIKGSTSGSGKPGSGEGSTKGQVQLVQSGSELKKPGASVKISCKASGYTFTSYAMNWVRQAPGQGLESMGWINTNTGNPTYAQGFTGRFVFSMDTSVSTAYLQISSLKAEDTAIYYCAPRYSSSWYLDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 20 aa, Artificial sequence
  Synthetic peptide
METDTLLLWVLLLWVPGSTG

SEQ ID NO: 5 - protein, 108 aa, Artificial sequence
  Synthetic peptide
EIVLTQSPATLSLSPGERATLSCRASQSVSSYLAWYQQKPGQAPRLLIYDASNRATGIPARFSGSGSGTDFTLTISSLEPEDFAVYYCQQRSNWLMYTFGQGTKLEIK

SEQ ID NO: 6 - protein, 11 aa, Artificial sequence
  Synthetic peptide
RASQSVSSYLA

SEQ ID NO: 7 - protein, 7 aa, Artificial sequence
  Synthetic peptide
DASNRAT

SEQ ID NO: 8 - protein, 10 aa, Artificial sequence
  Synthetic peptide
QQRSNWLMYT

SEQ ID NO: 9 - protein, 18 aa, Artificial sequence
  Synthetic peptide
GSTSGSGKPGSGEGSTKG

SEQ ID NO: 10 - protein, 119 aa, Artificial sequence
  Synthetic peptide
QVQLVQSGSELKKPGASVKISCKASGYTFTSYAMNWVRQAPGQGLESMGWINTNTGNPTYAQGFTGRFVFSMDTSVSTAYLQISSLKAEDTAIYYCAPRYSSSWYLDYWGQGTLVTVSS

SEQ ID NO: 11 - protein, 10 aa, Artificial sequence
  Synthetic peptide
GYTFTSYAMN

SEQ ID NO: 12 - protein, 17 aa, Artificial sequence
  Synthetic peptide
WINTNTGNPTYAQGFTG

SEQ ID NO: 13 - protein, 10 aa, Artificial sequence
  Synthetic peptide
RYSSSWYLDY

SEQ ID NO: 14 - protein, 12 aa, Artificial sequence
  Synthetic peptide
ESKYGPPCPPCP

SEQ ID NO: 15 - protein, 119 aa, Artificial sequence
  Synthetic peptide
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 229 aa, Artificial sequence
  Synthetic peptide
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 27 aa, Artificial sequence
  Synthetic peptide
FWVLVVVGGVLACYSLLVTVAFIIFWV

SEQ ID NO: 18 - protein, 41 aa, Artificial sequence
  Synthetic peptide
RSKRSRGGHSDYMNMTPRRPGPTRKHYQPYAPPRDFAAYRS

SEQ ID NO: 19 - protein, 112 aa, Artificial sequence
  Synthetic peptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 5 aa, Artificial sequence
  Synthetic peptide
DKTHT

SEQ ID NO: 21 - protein, 4 aa, Artificial sequence
  Synthetic peptide
CPPC

SEQ ID NO: 22 - protein, 15 aa, Artificial sequence
  Synthetic peptide
CPEPKSCDTPPPCPR

SEQ ID NO: 23 - protein, 12 aa, Artificial sequence
  Synthetic peptide
ELKTPLGDTTHT

SEQ ID NO: 24 - protein, 10 aa, Artificial sequence
  Synthetic peptide
KSCDKTHTCP

SEQ ID NO: 25 - protein, 7 aa, Artificial sequence
  Synthetic peptide
KCCVDCP

SEQ ID NO: 26 - protein, 7 aa, Artificial sequence
  Synthetic peptide
KYGPPCP

SEQ ID NO: 27 - protein, 15 aa, Artificial sequence
  Synthetic peptide
EPKSCDKTHTCPPCP

SEQ ID NO: 28 - protein, 12 aa, Artificial sequence
  Synthetic peptide
ERKCCVECPPCP

SEQ ID NO: 29 - protein, 17 aa, Artificial sequence
  Synthetic peptide
ELKTPLGDTTHTCPRCP

SEQ ID NO: 30 - protein, 12 aa, Artificial sequence
  Synthetic peptide
SPNMVPHAHHAQ

SEQ ID NO: 31 - protein, 12 aa, Artificial sequence
  Synthetic peptide
ESKYGPPCPPCP

SEQ ID NO: 32 - protein, 12 aa, Artificial sequence
  Synthetic peptide
ESKYGPPCPSCP

SEQ ID NO: 33 - protein, 15 aa, Artificial sequence
  Synthetic peptide
EPKSCDKTYTCPPCP

SEQ ID NO: 34 - protein, 45 aa, Artificial sequence
  Synthetic peptide
TTTPAPRPPTPAPTIASQPLSLRPEACRPAAGGAVHTRGLDFACD

SEQ ID NO: 35 - protein, 110 aa, Artificial sequence
  Synthetic peptide
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 36 - protein, 107 aa, Artificial sequence
  Synthetic peptide
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 23 aa, Artificial sequence
  Synthetic peptide
LGLLVAGVLVLLVSLGVAIHLCC

SEQ ID NO: 38 - protein, 25 aa, Artificial sequence
  Synthetic peptide
ALIVLGGVAGLLLFIGLGIFFCVRC

SEQ ID NO: 39 - protein, 23 aa, Artificial sequence
  Synthetic peptide
LCYLLDGILFIYGVILTALFLRV

SEQ ID NO: 40 - protein, 26 aa, Artificial sequence
  Synthetic peptide
WVLVVVGGVLACYSLLVTVAFIIFWV

SEQ ID NO: 41 - protein, 26 aa, Artificial sequence
  Synthetic peptide
VAAILGLGLVLGLLGPLAILLALYLL

SEQ ID NO: 42 - protein, 24 aa, Artificial sequence
  Synthetic peptide
ALPAALAVISFLLGLGLGVACVLA

SEQ ID NO: 43 - protein, 113 aa, Artificial sequence
  Synthetic peptide
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 44 - protein, 112 aa, Artificial sequence
  Synthetic peptide
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 45 - protein, 102 aa, Artificial sequence
  Synthetic peptide
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 46 - protein, 101 aa, Artificial sequence
  Synthetic peptide
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 47 - protein, 21 aa, Artificial sequence
  Synthetic peptide
ESPYQELQGQRSDVYSDLNTQ

SEQ ID NO: 48 - protein, 86 aa, Artificial sequence
  Synthetic peptide
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 49 - protein, 21 aa, Artificial sequence
  Synthetic peptide
DGVYTGLSTRNQETYETLKHE

SEQ ID NO: 50 - protein, 171 aa, Artificial sequence
  Synthetic peptide
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 51 - protein, 127 aa, Artificial sequence
  Synthetic peptide
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 52 - protein, 21 aa, Artificial sequence
  Synthetic peptide
DQVYQPLRDRDDAQYSHLGGN

SEQ ID NO: 53 - protein, 207 aa, Artificial sequence
  Synthetic peptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 54 - protein, 21 aa, Artificial sequence
  Synthetic peptide
NPDYEPIRKGQRDLYSGLNQR

SEQ ID NO: 55 - protein, 182 aa, Artificial sequence
  Synthetic peptide
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 56 - protein, 21 aa, Artificial sequence
  Synthetic peptide
DQLYQPLKDREDDQYSHLQGN

SEQ ID NO: 57 - protein, 163 aa, Artificial sequence
  Synthetic peptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 164 aa, Artificial sequence
  Synthetic peptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 59 - protein, 112 aa, Artificial sequence; identical to SEQ ID NO: 19
  Synthetic peptide

SEQ ID NO: 60 - protein, 112 aa, Artificial sequence; identical to SEQ ID NO: 19
  Synthetic peptide

SEQ ID NO: 61 - protein, 21 aa, Artificial sequence
  Synthetic peptide
NQLYNELNLGRREEYDVLDKR

SEQ ID NO: 62 - protein, 22 aa, Artificial sequence
  Synthetic peptide
EGLYNELQKDKMAEAYSEIGMK

SEQ ID NO: 63 - protein, 21 aa, Artificial sequence
  Synthetic peptide
DGLYQGLSTATKDTYDALHMQ

SEQ ID NO: 64 - protein, 226 aa, Artificial sequence
  Synthetic peptide
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 65 - protein, 188 aa, Artificial sequence
  Synthetic peptide
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 66 - protein, 21 aa, Artificial sequence
  Synthetic peptide
ENLYEGLNLDDCSMYEDISRG

SEQ ID NO: 67 - protein, 68 aa, Artificial sequence
  Synthetic peptide
FWVLVVVGGVLACYSLLVTVAFIIFWVRSKRSRLLHSDYMNMTPRRPGPTRKHYQPYAPPRDFAAYRS

SEQ ID NO: 68 - protein, 68 aa, Artificial sequence; identical to SEQ ID NO: 67
  Synthetic peptide

SEQ ID NO: 69 - protein, 619 aa, Artificial sequence
  Synthetic peptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 70 - protein, 42 aa, Artificial sequence
  Synthetic peptide
KRGRKKLLYIFKQPFMRPVQTTQEEDGCSCRFPEEEEGGCEL

SEQ ID NO: 71 - protein, 44 aa, Artificial sequence
  Synthetic peptide
FWVRSKRSRLLHSDYMNMTPRRPGPTRKHYQPYAPPRDFAAYRS

SEQ ID NO: 72 - protein, 35 aa, Artificial sequence
  Synthetic peptide
TKKKYSSSVHDPNGEYMFMRAVNTAKKSRLTDVTL

SEQ ID NO: 73 - protein, 37 aa, Artificial sequence
  Synthetic peptide
RRDQRLPPDAHKPPGGGSFRTPIQEEQADAHSTLAKI

SEQ ID NO: 74 - protein, 114 aa, Artificial sequence
  Synthetic peptide
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 75 - protein, 49 aa, Artificial sequence
  Synthetic peptide
HQRRKYRSNKGESPVEPAEPCRYSCPREEEGSTIPIQEDYRKPEPACSP

SEQ ID NO: 76 - protein, 187 aa, Artificial sequence
  Synthetic peptide
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 77 - protein, 54 aa, Artificial sequence
  Synthetic peptide
HIWQLRSQCMWPRETQLLLEVPPSTEDARSCQFPEEERGERSAEEKGRLGDLWV

SEQ ID NO: 78 - protein, 60 aa, Artificial sequence
  Synthetic peptide
CVKRRKPRGDVVKVIVSVQRKRQEAEGEATVIEALQAPPDVTTVAVEETIPSFTGRSPNH

SEQ ID NO: 79 - protein, 9 aa, Artificial sequence
  Synthetic peptide
YPYDVPDYA

SEQ ID NO: 80 - protein, 8 aa, Artificial sequence
  Synthetic peptide
DYKDDDDK

SEQ ID NO: 81 - protein, 10 aa, Artificial sequence
  Synthetic peptide
EQKLISEEDL

SEQ ID NO: 82 - protein, 4 aa, Artificial sequence
  Synthetic peptide
GGSG

SEQ ID NO: 83 - protein, 5 aa, Artificial sequence
  Synthetic peptide
GGSGG

SEQ ID NO: 84 - protein, 5 aa, Artificial sequence
  Synthetic peptide
GSGSG

SEQ ID NO: 85 - protein, 5 aa, Artificial sequence
  Synthetic peptide
GSGGG

SEQ ID NO: 86 - protein, 5 aa, Artificial sequence
  Synthetic peptide
GGGSG

SEQ ID NO: 87 - protein, 5 aa, Artificial sequence
  Synthetic peptide
GSSSG

SEQ ID NO: 88 - protein, 675 aa, Artificial sequence
  Synthetic polypeptide
METDTLLLWVLLLWVPGSTGEIVLTQSPATLSLSPGERATLSCRASQSVSSYLAWYQQKPGQAPRLLIYDASNRATGIPARFSGSGSGTDFTLTISSLEPEDFAVYYCQQRSNWLMYTFGQGTKLEIKGSTSGSGKPGSGEGSTKGQVQLVQSGSELKKPGASVKISCKASGYTFTSYAMNWVRQAPGQGLESMGWINTNTGNPTYAQGFTGRFVFSMDTSVSTAYLQISSLKAEDTAIYYCAPRYSSSWYLDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 9 aa, Artificial sequence
  Synthetic polypeptide
CPYSNPSLC

(19) United States
(12) Patent Application Publication
(10) Pub. No.: US 2023/0049954 A1
(43) Pub. Date: Feb. 16, 2023

(54) CHIMERIC ANTIGEN RECEPTORS AND RELATED METHODS AND COMPOSITIONS FOR THE TREATMENT OF CANCER

(71) Applicant: THE REGENTS OF THE UNIVERSITY OF CALIFORNIA, Oakland, CA (US)

(72) Inventors: Cristina PUIG-SAUS, Los Angeles, CA (US); Antoni RIBAS, Los Angeles, CA (US); Yvonne CHEN, Los Angeles, CA (US)

(73) Assignee: THE REGENTS OF THE UNIVERSITY OF CALIFORNIA, Oakland, CA (US)

(21) Appl. No.: 17/640,281

(22) PCT Filed: Sep. 4, 2020
(86) PCT No.: PCT/US2020/049521

§ 371 Date: Mar. 3, 2022

Related U.S. Application Data
(60) Provisional application No. 62/897,062, filed on Sep. 6, 2019.

(51) Int. Cl. A61K 35/17; C07K 14/705; C07K 14/725; A61P 35/00; C07K 16/28; A61P 17/00

(52) U.S. Cl. CPC A61K 35/17; C07K 14/70521; C07K 14/7051; C07K 14/70578; C07K 14/70596; A61P 35/00; C07K 16/2803; A61P 17/00; C07K 2319/03; C07K 2319/02; C07K 2317/526; C07K 2317/524; C07K 2317/53; A61K 2039/505

(57) ABSTRACT

Methods and compositions are provided related to therapeutic receptors, including chimeric antigen receptors (CARs), capable of specifically binding TYRP-1. The disclosed compositions include, for example, cells (e.g., immune cells) expressing TYRP-1 specific CARs, nucleic acids encoding TYRP-1 specific CARs, and TYRP-1 specific CAR polypeptides. Certain aspects relate to methods of treating cancer, including melanoma, using compositions comprising TYRP-1 specific CARs, for example cells expressing TYRP-1 specific CARs. In some embodiments, provided herein are chimeric polypeptides comprising a TYRP-1 binding domain, a hinge region, a transmembrane domain, and an intracellular signaling domain.

### CROSS-REFERENCE

This application claims priority to U.S. Provisional Patent Application No. 62/897,062, filed Sep. 6, 2019, which is hereby incorporated by reference in its entirety.

### BACKGROUND

This invention was made with government support under Grant Number CA197633, awarded by the National Institutes of Health. The government has certain rights in the invention.

### FIELD OF THE INVENTION

This invention relates generally to the fields of molecular biology and immunotherapy.

### BACKGROUND

Melanoma is a cancer of the skin and accounts for over 96,000 new cancer diagnoses each year. Immune checkpoint blockade (ICB) has been approved as front line therapy for advanced metastatic melanoma. Despite the clinical responses achieved by ICB, with response rates of about 60% with anti-CTLA-4 and anti-PD-1 combination therapy, a large proportion of patients do not respond to treatment and some responders relapse. Thus, there is a need for therapies suitable for melanoma patients who are unresponsive or refractory to ICB.

Tyrosinase-related protein-1 (TYRP-1) is a transmembrane glycoprotein that is specifically expressed in melanocytes and melanoma cells. TYRP-1 is widely expressed in melanoma tumors, and its expression at high levels is associated with unfavorable outcomes. Recognized herein is a need for compositions and methods for effective targeting of TYRP-1 for the treatment of melanoma.

### SUMMARY OF THE DISCLOSURE

The current disclosure fulfills the need in the art for therapeutic receptors, including chimeric antigen receptors (CARs), that target TYRP-1 for the treatment cancer. Accordingly, certain aspects of this disclosure relate to treating melanoma. Further embodiments relate to antigen binding domains targeting TYRP-1 comprising a sequence with at least 90% sequence identity to SEQ ID NO:5 or SEQ ID NO:10. Compositions and methods concerning polypeptides that are therapeutic receptors binding TYRP-1 are provided as a solution for treating cancer, such as melanoma. Embodiments include TYRP-1 targeted polypeptides, nucleic acids encoding a TYRP-1 targeted polypeptide, vectors comprising nucleic acids encoding a TYRP-1 targeted polypeptide, cells comprising nucleic acids or vectors encoding a TYRP-1 targeted polypeptide, cells expressing a TYRP-1 targeted polypeptide on their surface, pharmaceutical compositions comprising a TYRP-1 targeted polypeptide, pharmaceutical compositions comprising a cell expressing a TYRP-1 targeted polypeptide, methods of making a TYRP-1 targeted polypeptide, methods of making T cells expressing a TYRP-1 targeted polypeptide, methods of treating a subject with a composition comprising a TYRP-1 targeted polypeptide, populations of cells comprising TYRP-1 targeted polypeptides, and polypeptides comprising a TYRP-1 targeted antigen binding domain. It is specifically contemplated that one or more of these elements may be excluded from certain embodiments of the disclosure.

In some embodiments, the CAR molecules discussed herein have the three main regions of a CAR molecule, which are an extracellular domain that binds to one or more target molecule(s), a cytoplasmic region that contains a primary intracellular signaling domain, and a transmembrane region between the extracellular domain and the cytoplasmic domain. Some CAR molecules have a spacer that is between the extracellular domain and the transmembrane domain. Furthermore, one or more linkers may be included in CAR molecules between or within one or more regions, such as between different binding regions within the extracellular domain or within a binding region, such as between the variable region of a light chain (VH) and the variable region of a heavy chain (VL). One or more tags may be included in CAR molecules of the disclosure. A tag may be between or within one or more regions. For example, a CAR molecule may comprise a tag between a VH region and a VL region. In another example, a CAR molecule may comprise a tag between two different antigen binding regions. In a further example, a CAR molecule may comprise a tag at the N-terminus of the molecule. Any embodiment regarding a specific region may be implemented with respect to any other specific region disclosed herein. Examples of regions which can be implemented with any other specific region include, but are not limited to the following: extracellular domain, TYRP-1 targeted domain, VH domain having at least 90% sequence identity with SEQ ID NO:10, Vh domain comprising SEQ ID NO:10, VL domain having at least 90% sequence identity with SEQ ID NO:5, VL domain comprising SEQ ID NO:5, linker, hinge, extracellular spacer, transmembrane domain, cytoplasmic domain, intracellular signaling domain, primary intracellular signaling domain, costimulatory domain, tag, detection peptide, and leader peptide. One or more of these regions may be excluded from certain embodiments of the disclosure. Any of these regions may be immediately adjacent either on the N-terminal side or the C-terminal side of another region depending on its function but it is also contemplated that there may be intervening amino acids between contiguous regions that are at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 amino acids in length (or any range derivable therein).

Further aspects relate to a chimeric polypeptide comprising (a) an antigen binding domain comprising (i) a variable heavy (VH) region; and (ii) a variable light (VL) region; a transmembrane domain, and an intracellular signaling domain. In some embodiments, the VH region has at least 90% sequence identity with SEQ ID NO:10. In some embodiments, the VH region comprises SEQ ID NO:11 (HCDR1), SEQ ID NO:12 (HCDR2), and SEQ ID NO:13 (HCDR3). In some embodiments, the VL region has at least 90% sequence identity with SEQ ID NO:5. In some embodiments, the VL region comprises SEQ ID NO:6 (LCDR1), SEQ ID NO:7 (LCDR2), and SEQ ID NO:8 (LCDR3).

Method aspects of the disclosure relate to the use of the CAR molecules, compositions, and cells of the disclosure for the treatment of cancer. In some embodiments, the cancer is a skin cancer. In some embodiments, the cancer comprises a TYRP-1+ cancer, wherein a TYRP-1+ cancer is one that comprises TYRP-1+ cells or comprises at least 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, or 90% (or any range derivable therein) TYRP-1+ cancer cells in a population of tumor cells. In some embodiments, the cancer comprises melanoma. The CAR polypeptides of the current disclosure may have a region, domain, linker, spacer, or other portion thereof that comprises or consists of an amino acid sequence that is at least, at most, or exactly 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) to all or a portion of the amino acid sequences described herein. In certain embodiments, a CAR polypeptide comprises or consists of an amino acid sequence that is, is at least, is at most, or exactly 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identical (or any range derivable therein) to any one of SEQ ID NOS: 1-89.

In some embodiments, the polypeptides of the present disclosure comprise a VH domain and a VL domain. In some embodiments, the VH and VL domains are separated by a linker. In one embodiment, the order of the variable regions is VH-VL. In another embodiment, the order of the variable regions is VL-VH. It is contemplated that a polypeptide may comprise multiple linkers such as 1, 2, 3, 4, 5 or more linkers. The linker is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 amino acids (or any range derivable therein) in length. In certain embodiments, the linker is 4-40 amino acids in length. It is contemplated that a linker may separate any domain/region in the CAR polypeptides described herein. In some embodiments, the linker is composed of only glycine and serine residues (a glycine-serine linker). In some embodiments, the linker is a linker having sequence GSTSGSGKPGSGEGSTKG (SEQ ID NO: 9).

“Single-chain Fv” or “scFv” antibody fragments comprise at least a portion of the VH and VL domains of an antibody, such as the CDRs of each, wherein these domains are present in a single polypeptide chain. It is contemplated that an scFv includes a CDR1, CDR2, and/or CDR3 of a heavy chain variable region and a CDR1, CDR2, and/or CDR3 of a light chain variable region in some embodiments. It is further contemplated that a CDR1, CDR2, or CDR3 may comprise or consist of a sequence set forth in a SEQ ID NO provided herein as CDR1, CDR2, or CDR3, respectively. A CDR may also comprise 1, 2, 3, 4, 5, 6, 7, 8, 9, 10 or more contiguous amino acid residues (or any range derivable therein) flanking one or both sides of a particular CDR sequence; therefore, there may be one or more additional amino acids at the N-terminal or C-terminal end of a particular CDR sequence, such as those shown in Tables 1-3.

It is also contemplated that an scFv may comprise more than the CDRs of a light chain variable region and/or a heavy chain region. In some embodiments, all or part of a light chain variable region and/or all or part of a heavy chain variable region is included in an scFv that is part of a binding domain. In some embodiments, the order is VH-VL, while in other embodiments, the order is VL-VH. Moreover, a VH, VL, VH-VL, or VL-VH sequence provided herein may have 1, 2, 3, 4, 5, 6, 7, 8, 9, 10 or more additions, deletions, and/or substitutions, particularly if such changes do not alter the CDRs of the light and heavy variable chain regions.

In some embodiments, CAR molecules of the present disclosure comprise a transmembrane domain between the extracellular domain and the cytoplasmic region (also referred to as an intracellular domain). Embodiments include a transmembrane domain that is an alpha or beta chain of the T cell receptor, CD28, CD3 epsilon, CD45, CD4, CD5, CD8, CD9, CD16, CD22, CD33, CD37, CD64, CD80, CD86, CD123, CD134, CD137 or CD154 transmembrane domain. In some embodiments, the transmembrane domain is a CD28 transmembrane domain. In some embodiments, the transmembrane domain comprises SEQ ID NO:17.

In some embodiments, CAR molecules of the present disclosure have a cytoplasmic region that mediates internal cell signaling. In some embodiments, this is accomplished with the signaling domain from CD3ζ (zeta), which acts as a primary or main intracellular. In some embodiments, an intracellular signaling domain comprises a primary signaling domain comprises SEQ ID NO:19. A cytoplasmic region includes 1, 2, or 3 costimulatory domains in further embodiments. In some embodiments, a cytoplasmic region comprises two costimulatory domains. In certain embodiments, a costimulatory domain is from 4-1BB (CD137), CD28, IL-15Rα, OX40, CD2, CD27, CDS, ICAM-1, LFA-1 (CD11a/CD18), or ICOS (CD278), though other costimulatory domains may also be included. In certain embodiments, the costimulatory domain is a 4-1BB costimulatory domain. In some embodiments, the costimulatory domain comprises SEQ ID NO:70. In some embodiments, the costimulatory domain is a CD28 costimulatory domain. In some embodiments, the costimulatory domain comprises SEQ ID NO:18.

In certain embodiments, polypeptides described throughout this disclosure are isolated, meaning they are not found in the cellular milieu. In some cases, they are purified, which means it is mostly if not completely separated from polypeptides having a different amino acid sequence and/or chemical formula.

Nucleic acids comprising a sequence that encodes the chimeric antigen receptors disclosed herein, and portions thereof, are provided in embodiments. A nucleic acid may comprise RNA or DNA. In certain embodiments, the nucleic acid is an expression construct. In some embodiments, the expression construct is a vector. In certain embodiments, the vector is a viral vector. The viral vector is a retroviral vector or derived from a retrovirus in particular embodiments. In some embodiments, the retroviral vector comprises a lentiviral vector or is derived from a lentivirus. It is noted that a viral vector is an integrating nucleic acid in certain embodiments. Additionally, a nucleic acid may be a molecule involved in gene editing such that a nucleic acid (e.g. DNA, RNA) encoding a CAR is used to incorporate a CAR-coding sequence into a particular locus of the genome, such as the TRAC locus. This involves a gene editing system such as CRISPR/Cas9 in some embodiments. A nucleic acid, polynucleotide, or polynucleotide region (or a polypeptide or polypeptide region) has a certain percentage (for example, 60%, 65%, 70%, 75%, 80%, 85%, 90%, 95%, 98% or 99%—or any range derivable therein) of “sequence identity” or “homology” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. It is contemplated that a nucleic acid may have such sequence identity or homology to any nucleic acid SEQ ID NO provided herein.

In other embodiments, there is a cell or a population of cells comprising a nucleic acid that encodes all or part of any CAR discussed herein. In certain embodiments, a cell or population of cells contains within its genome a sequence encoding any of the CAR polypeptides described herein. This includes, but is not limited to, a lentivirus or retrovirus that has integrated into the cell's genome. In some embodiments, a cell or population of cells expresses all or part of any CAR discussed herein, including, but not limited to those with the amino acid sequence of any of SEQ ID NO: 1-89. Progeny (F1, F2, and beyond) of cells in which a nucleic acid encoding a CAR polypeptide was introduced are included in the cells or populations of cells disclosed herein. In some embodiments, a cell or population of cells is a T cell, a natural killer (NK) cell, a natural killer T cell (NKT), an invariant natural killer T cell (iNKT), stem cell, lymphoid progenitor cell, peripheral blood mononuclear cell (PBMC), peripheral blood stem cell (PBSC), bone marrow cell, fetal liver cell, embryonic stem cell, cord blood cell, induced pluripotent stem cell (iPS cell). Specific embodiments concern a cell that is a T cell or an NK cell. In some embodiments, T cell comprises a naïve memory T cell. In some embodiments, the naïve memory T cell comprises a CD4+ or CD8+ T cell. In some embodiments, the cells are a population of cells comprising both CD4+ and CD8+ T cells. In some embodiments, the cells are a population of cells comprising naïve memory T cells comprising CD4+ and CD8+ T cells. In some embodiments, the T cell comprises a T cell from a population of CD14 depleted, CD25 depleted, and/or CD62L enriched PBMCs. In embodiments involving a population of cells, the population is about, is at least about, or is at most about 102, 10³, 10⁴, 10⁵, 10⁶, 10⁷, 10⁸, 10⁹, 10¹⁰, 10¹¹, 10¹² cells (or any range derivable therein. In certain embodiments, there are about 10³-10⁸ cells. In certain embodiments, cells are autologous with respect to a patient who will receive them. In other embodiments, cells are not autologous and may be allogenic.

In some aspects, the disclosure relates to a cell comprising one or more polypeptides described herein. In some embodiments, the cell is an immune cell. In some embodiments, the cell is a progenitor cell or stem cell. In some embodiments, the progenitor or stem cell is in vitro differentiated into an immune cell. In some embodiments, the cell is a T cell. In some embodiments, the cell is a CD4+ or CD8+ T cell. In some embodiments, the cell is a natural killer cell. In some embodiments, the cell is ex vivo. The term immune cells includes cells of the immune system that are involved in defending the body against both infectious disease and foreign materials. Immune cells may include, for example, neutrophils, eosinophils, basophils, natural killer cells, lymphocytes such as B cells and T cells, and monocytes. T cells may include, for example, CD4+, CD8+, T helper cells, cytotoxic T cells, γδ T cells, regulatory T cells, suppressor T cells, Th1 cells, Th2 cells, Th17 cells, and natural killer T cells. In a specific embodiment, the T cell is a regulatory T cell.

Also included as an embodiment is a composition comprising the population of cells, wherein the composition is a pharmaceutically acceptable formulation.

Methods of making and using the chimeric antigen receptors, nucleic acids encoding such CARs, and cells and compositions containing these CARs are also provided. Methods include methods for making a cell that expresses a CAR, for treating a patient with cancer, for treating a patient with melanoma, for developing a T cell or an NK cell that expresses a CAR, for expressing a TYRP-1 targeted CAR molecule.

Steps of methods include 1, 2, 3, 4, 5, 6, 7, 8, 9, 10 or more of the following steps: cloning regions of a TYRP-1 specific CAR; introducing into a cell a nucleic acid that encodes a TYRP-1 specific CAR; editing the genome of a cell to express a TYRP-1 specific CAR; infecting a cell with a viral vector encoding a TYRP-1 specific CAR; introducing a guide RNA (gRNA) and/or a template into a cell for editing a genome to express a TYRP-1 specific CAR; culturing a cell or a population of cells; expanding a cell or a population of cells; differentiating a cell or a population of cells into a cell with one or more T cell or NK cell properties; culturing a cell with serum-free medium; culturing a cell under conditions to produce a T cell or NK cell; purifying cells that express TYRP-1 specific CARs; administering cells expressing a TYRP-1 specific CAR to a patient; obtaining cells from a patient; isolating cells from a patient; selecting cells that express a TYRP-1 specific CAR; isolating cells using a sortable tag; detecting a tag associated with a TYRP-1 specific CAR; measuring a tag associated with a TYRP-1 specific CAR; or administering other cancer therapy to a patient in addition to administering cells that express TYRP-1 specific CAR molecules.

In certain embodiments, there are methods of making a cell that expresses a chimeric antigen receptor comprising introducing into a cell a nucleic acid encoding one of the CAR molecules discussed herein or a nucleic acid that allows gene editing of the cell's genome to express one of the CAR molecules discussed herein. In certain embodiments, a cell is transduced with a lentivirus encoding the CAR. In some embodiments, a cell is transduced with a retrovirus encoding the CAR. In some embodiments a cell is a T cell, a natural killer (NK) cell, a natural killer T cell (NKT), an invariant natural killer T cell (iNKT), stem cell, lymphoid progenitor cell, peripheral blood mononuclear cell (PBMC), peripheral blood stem cell (PBSC), bone marrow cell, fetal liver cell, embryonic stem cell, cord blood cell, induced pluripotent stem cell (iPS cell). In cases where a cell is not yet a T cell or NK cell, a method may also include culturing the cell under conditions that promote the differentiation of the cell into a T cell or an NK cell. In additional embodiments, methods include culturing the cell under conditions to expand the cell before and/or after introducing the nucleic acid into the cell. In some embodiments, cells are cultured with serum-free medium.

Additional methods concern treating a patient with cancer comprising administering to the patient an effective amount of a composition comprising a cell population expressing a TYRP-1 targeted CAR. In some embodiments, the patient has a skin cancer. In particular embodiments, a patient has melanoma. In additional embodiments, a patient has relapsed melanoma. Further embodiments include a step of administering an additional therapy to the patient. Further embodiments include a step of administering chemotherapy and/or radiation to the patient. In some embodiments, the additional therapy comprises an immunotherapy. In some embodiments, the additional therapy comprises an additional therapy described herein. In some embodiments, the immunotherapy comprises immune checkpoint inhibitor therapy. In some embodiments, the immunotherapy comprises an immunotherapy described herein. In some embodiments, the immune checkpoint inhibitor therapy comprises a PD-1 inhibitor and/or CTLA-4 inhibitor. In some embodiments, the immune checkpoint inhibitor therapy comprises one or more inhibitors of one or more immune checkpoint proteins described herein.

In some embodiments, disclosed herein is a chimeric polypeptide comprising (a) an antigen binding domain comprising (i) a variable heavy (VH) region having at least 90% sequence identity with SEQ ID NO:10 and (ii) a variable light (VL) region having at least 90% sequence identity with SEQ ID NO:5, (b) a transmembrane domain, and (c) an intracellular domain. In some embodiments, the VH region comprises SEQ ID NO:10. In some embodiments, the VL region comprises SEQ ID NO:5. In some embodiments, the chimeric polypeptide further comprises a signal peptide. In some embodiments, the chimeric polypeptide further comprises a hinge region. In some embodiments, the VH and VL regions are separated by a linker, for example a linker between 4 and 40 amino acids in length. In some embodiments, the linker comprises SEQ ID NO:9. In some embodiments, the linker comprises SEQ ID NO:89. In some embodiments, the transmembrane domain is an alpha or beta chain of the T cell receptor or a transmembrane domain from CD28, CD3ε (epsilon), CD45, CD4, CD5, CD8, CD9, CD16, CD22, CD33, CD37, CD64, CD80, CD86, CD123, CD134, CD137 or CD154. In some embodiments, the transmembrane domain comprises SEQ ID NO:17. In some embodiments, the intracellular signaling domain comprises a CD3ζ (zeta) signaling domain. In some embodiments, the intracellular signaling domain comprises SEQ ID NO:19. In some embodiments, the intracellular signaling domain comprises a signaling domain from a cytokine receptor. In some embodiments, the intracellular signaling domain comprises a signaling domain from 4-1BB (CD137), CD28, IL-15Rα, OX40, CD2, CD27, CDS, ICAM-1, LFA-1 (CD11a/CD18), or ICOS (CD278). In some embodiments, the intracellular signaling domain comprises SEQ ID NO:18. In some embodiments, the intracellular signaling domain comprises SEQ ID NO:70. In some embodiments, the antigen binding domain and the transmembrane domain are separated by a hinge region. In some embodiments, the hinge region comprises an IgG4 hinge, a CD8α hinge, an IgG1 hinge, or a CD34 hinge. In some embodiments, the hinge region comprises SEQ ID NO:14. In some embodiments, the hinge region comprises SEQ ID NO:15. In some embodiments, the hinge region comprises SEQ ID NO:16.

In some aspects, disclosed herein is a chimeric polypeptide comprising (a) a signal peptide, (b) an antigen binding domain comprising (i) a variable heavy region comprising SEQ ID NO:10 and (ii) a variable light region comprising SEQ ID NO:5; (c) a hinge region between 8 and 300 amino acids in length; (d) a transmembrane domain; and (e) an intracellular domain. In some embodiments, the transmembrane domain is a CD28 transmembrane domain. In some embodiments, the intracellular signaling domain comprises a CD28 signaling domain and a CD3ζ (zeta) signaling domain. In some embodiments, the VH region and the VL region are separated by a linker. In some embodiments, the linker comprises SEQ ID NO: 9. In some embodiments, the hinge region comprises SEQ ID NO:14. In some embodiments, the hinge region comprises SEQ ID NO:15. In some embodiments, the hinge region comprises SEQ ID NO:16. In some embodiments, the chimeric polypeptide comprises SEQ ID NO:1. In some embodiments, the chimeric polypeptide comprises SEQ ID NO:2. In some embodiments, the chimeric polypeptide comprises SEQ ID NO:3. In some embodiments, the chimeric polypeptide comprises SEQ ID NO:88. In some embodiments, the antigen binding domain specifically binds to a TYRP-1 protein.

Disclosed herein, in some embodiments, is a nucleic acid encoding any of the chimeric polypeptides (e.g., CARs) described herein. In some embodiments, the nucleic acid is an expression construct. In some embodiments, the expression construct is a plasmid. In some embodiments, the expression construct is a viral vector. In some embodiments, the viral vector is a vector derived from a retrovirus or a vector derived from a lentivirus. Disclosed herein, in some aspects, is a cell comprising any of the nucleic acids described herein. In some embodiments, the nucleic acid is integrated into a genome of the cell. Disclosed herein, in some aspects, is a cell comprising any of the chimeric polypeptides (e.g., CARs) described herein.

In some embodiments, the cell is an immune cell. In some embodiments, the cell is a T cell, a natural killer (NK) cell, a natural killer T cell (NKT), an invariant natural killer T cell (iNKT), a stem cell, a lymphoid progenitor cell, a peripheral blood mononuclear cell (PBMC), a peripheral blood stem cell (PBSC), a bone marrow cell, a fetal liver cell, an embryonic stem cell, a cord blood cell, or an induced pluripotent stem cell (iPS cell). In some embodiments, the cell is a memory T cell. Disclosed herein, in some embodiments, is a population of cells comprising a cell disclosed herein. Further embodiments pertain to a pharmaceutical composition comprising a population of cells.

The present disclosure provides, in some embodiments, a method for treating a subject with cancer comprising administering to the subject an effective amount of a population of cells or pharmaceutical composition comprising a chimeric polypeptide or nucleic acid encoding a chimeric polypeptide.

Use of the one or more sequences or compositions may be employed based on any of the methods described herein. Other embodiments are discussed throughout this application. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Example section are understood to be embodiments that are applicable to all aspects of the technology described herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B show in vitro cytotoxicity of T cells expressing the shown CAR constructs against human melanoma cell lines with high expression of TYRP-1 (FIG. 1A) and human melanoma cell lines with low expression of TYRP-1 (FIG. 1B).

FIG. 2 shows in vitro cytokine secretion by human PBMCs expressing the shown CAR constructs upon co-culture with human melanoma cell lines having either high expression of TYRP-1 (top), low expression of TYRP-1 (lower left), or negative expression of TYRP-1 (lower right).

FIGS. 3A and 3B show in vitro cytotoxicity (FIG. 3A) and cytokine secretion (FIG. 3B) of mouse CD3+ T cells expressing the shown CAR constructs against the murine melanoma cell line B16-F10.

FIG. 4 shows in vivo antitumor activity of T cells expressing the 20D7SS, 20D7SM, or 20D7SL CAR constructs in C57/B6 immunocompetent mice bearing B16-F10 melanoma tumors.

FIG. 5 shows in vivo antitumor activity of different doses of murine T cells expressing the 20D7SL CAR construct in C56/B6 immunocompetent mice bearing B16-F10 melanoma tumors.

FIG. 6 shows in vivo antitumor activity of treatment with murine T cells expressing the 20D7SL CAR alone or in combination with standard IL-2 treatment in C57/B6 immunocompetent mice bearing B16-F10 melanoma tumors.

FIGS. 7A and 7B shows in vivo antitumor activity of human T cells expressing the 20D7SL CAR in patient-derived melanoma models in immunodeficient mouse models.

FIGS. 8A-8C shows in vitro cytotoxicity over time of the T cells expressing the 20D7SL CAR in a panel of human non-melanoma cell lines with negative expression of TYRP-1.

FIGS. 9A and 9B demonstrate a loss of in vitro cytokine secretion and cytotoxicity upon co-culture with TYRP-1 knockout cell lines.

FIGS. 10A and 10B show in vitro antitumor activity of T cells expressing a CARs comprising different costimulatory domains.

FIGS. 11A and 11B show in vivo antitumor activity of the T cells expressing CARS comprising different costimulatory signaling domains in patient-derived melanoma models in immunodeficient mouse models

FIGS. 12A-12C show TYRP-1 expression in all patients combining TCGA dataset, BMS-CA029, and MK3475-001 clinical trial datasets.

### DESCRIPTION OF ILLUSTRATIVE EMBODIMENTS

Disclosed herein are therapeutic receptors, including chimeric antigen receptors (CARs), capable of targeting TYRP-1 for the treatment of cancer. In some embodiments, CARs of the present disclosure are used in the treatment of melanoma. Using modular DNA assembly and high-throughput characterization methods, the inventors developed a panel of CARs with varying targeting efficiencies in response to TYRP-1, allowing the selection of the ideal construct that achieves therapeutic efficacy while avoiding potential toxicity against healthy tissue. Embodiments of the present disclosure are directed to CARs named 20D7SS (SEQ ID NO:1), 20D7SM (SEQ ID NO:2), and 20D7SL (SEQ ID NO:3), which differ in length of the hinge region. TYRP-1 targeted CARs are able to effectively target a variety of patient-derived melanoma cell lines and have varying degrees of targeting efficacy based on level of TYRP-1 expression on the surface of target cells. TYRP-1 CARs are selective of TYRP-1 and do not show cytotoxicity or cytokine release in cells with negative expression of TYRP-1. TYRP-1 targeted CARs are also capable of exerting control of established melanoma in immunocompetent mice with and without IL-2, and in different patient-derived melanoma models in immunocompromised mice. Additionally, TYRP-1 CARs with different intracellular signaling domains have similar antitumor activity in vitro and in vivo.

### I. Definitions

The peptides of the disclosure relate to peptides comprising chimeric antigen receptors, or CARs. CARs are engineered receptors, which are capable of grafting an arbitrary specificity onto an immune effector cell. In some cases, these receptors are used to graft the specificity of a monoclonal antibody onto a T cell. The receptors are called chimeric because they are composed of parts from different sources.

The terms “protein,” “polypeptide,” and “peptide” are used interchangeably herein when referring to a gene product.

“Homology,” or “identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 60% identity, less than 50% identity, less than 40% identity, less than 30% identity, or less than 25% identity, with one of the sequences of the current disclosure.

The terms “amino portion,” “N-terminus,” “amino terminus,” and the like as used herein are used to refer to order of the regions of the polypeptide. Furthermore, when something is N-terminal to a region it is not necessarily at the terminus (or end) of the entire polypeptide, but just at the N-terminus of the region or domain. Similarly, the terms “carboxy portion,” “C-terminus,” “carboxy terminus,” and the like as used herein is used to refer to order of the regions of the polypeptide, and when something is C-terminal to a region it is not necessarily at the terminus (or end) of the entire polypeptide, but just at the C-terminus of the region or domain.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof” means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The term functional antibody fragment includes antigen binding fragments of antibodies, including e.g., Fab′, F(ab′)2, Fab, Fv, rlgG, and scFv fragments. The term scFv refers to a single chain Fv antibody in which the variable domains of the heavy chain and of the light chain of a traditional two chain antibody have been joined to form one chain.

As used herein, the term “binding affinity” refers to the equilibrium constant for the reversible binding of two agents and is expressed as a dissociation constant (Kd). Binding affinity can be at least 25% greater, at least 50% greater, at least 75% greater, at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more (or any derivable range therein), than the binding affinity of an antibody for unrelated amino acid sequences. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges.

“Individual, “subject,” and “patient” are used interchangeably and can refer to a human or non-human.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

Throughout this application, the term “about” is used to indicate that a value includes the standard deviation of error for the device and/or method being employed to determine the value.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention. With respect to pharmaceutical compositions, the term “consisting essentially of” includes the active ingredients recited, excludes any other active ingredients, but does not exclude any pharmaceutical excipients or other components that are not therapeutically active.

The term “consisting of” refers to compositions, methods, and respective components thereof as described herein, which are exclusive of any element not recited in that description of the embodiment.

It is contemplated that embodiments described in the context of the term “comprising” may also be implemented in the context of the term “consisting of” or “consisting essentially of.”

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

### II. Polypeptides

A. Signal Peptide

Polypeptides of the present disclosure may comprise a signal peptide. A “signal peptide” refers to a peptide sequence that directs the transport and localization of the protein within a cell, e.g. to a certain cell organelle (such as the endoplasmic reticulum) and/or the cell surface. In some embodiments, a signal peptide directs the nascent protein into the endoplasmic reticulum. This is essential if a receptor is to be glycosylated and anchored in the cell membrane. Generally, the signal peptide natively attached to the amino-terminal most component is used (e.g. in an scFv with orientation light chain-linker-heavy chain, the native signal of the light-chain is used). In some embodiments the signal peptide comprises SEQ ID NO:4.

In some embodiments, the signal peptide is cleaved after passage of the endoplasmic reticulum (ER), i.e. is a cleavable signal peptide. In some embodiments, a restriction site is at the carboxy end of the signal peptide to facilitate cleavage.

B. Antigen Binding Domain

Polypeptides of the present disclosure may comprise one or more antigen binding domains. In some embodiments, a polypeptide comprises a TYRP-1 binding domain. In some embodiments, a polypeptide comprises a TYRP-1 binding domain and one or more additional binding domains. An “antigen binding domain” describes a region of a polypeptide capable of binding to an antigen under appropriate conditions. In some embodiments, an antigen binding domain is a single-chain variable fragment (scFv) based on one or more antibodies. In some embodiments, an antigen binding domain of polypeptides of the present disclosure is a scFv based on a TYRP-1 antibody, for example IMC-20D7S or any other TYRP-1 antibody. In some embodiments, an antigen binding domain comprises a variable heavy (VH) region and a variable light (VL) region, with the VH and VL regions being on the same polypeptide. In some embodiments, the antigen binding domain comprises a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by various techniques (e.g., site-directed mutagenesis or PCR-mediated mutagenesis) and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10⁻⁵ M, 10⁻⁶M, 10⁻⁷M, 10⁻⁸ M. 10⁻⁹ M, 10⁻¹⁰ M, 10⁻¹¹ M, 10⁻¹² M, or 10⁻¹³M. In some embodiments, the K_{D} of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰ M, 10⁻¹¹ M, 10⁻¹² M, or 10⁻¹³ M (or any derivable range therein).

Binding affinity, K_{A}, or K_{D} can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some embodiments, the TYRP-1-binding region is humanized. In some embodiments, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% (or any range derivable therein) binding affinity and/or expression level in host cells, compared to a polypeptide comprising a non-humanized binding region, such as a binding region from a mouse.

In some embodiments, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some embodiments, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

C. Extracellular Spacer

An extracellular spacer may link an antigen-binding domain to a transmembrane domain. In some embodiments, a hinge is flexible enough to allow the antigen-binding domain to orient in different directions to facilitate antigen binding. In one embodiment, the spacer is the hinge region from IgG. Alternatives include the CH2CH3 region of immunoglobulin and portions of CD3. In some embodiments, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some embodiments, the spacer is from IgG4. An extracellular spacer may comprise a hinge region.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgGl) is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 110, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some embodiments, the extracellular spacer consists of or comprises a hinge region from an immunoglobulin (e.g. IgG). Immunoglobulin hinge region amino acid sequences are known in the art; see, e.g., Tan et al. (1990) Proc. Natl. Acad. Sci. USA 87: 162; and Huck et al. (1986) Nucl. Acids Res.

The length of an extracellular spacer may have effects on the CAR's signaling activity and/or the CAR-T cells' expansion properties in response to antigen-stimulated CAR signaling. In some embodiments, a shorter spacer such as less than 50, 45, 40, 30, 35, 30, 25, 20, 15, 14, 13, 12, 11, or 10 amino acids is used. In some embodiments, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

As non-limiting examples, an immunoglobulin hinge region can include one of the following amino acid sequences: DKTHT (SEQ ID NO:20); CPPC (SEQ ID NO:21); CPEPKSCDTPPPCPR (SEQ ID NO:22); ELKTPLGDTTHT (SEQ ID NO:23); KSCDKTHTCP (SEQ ID NO:24); KCCVDCP (SEQ ID NO:25); KYGPPCP (SEQ ID NO:26); EPKSCDKTHTCPPCP (SEQ ID NO:27—human IgG1 hinge); ERKCCVECPPCP (SEQ ID NO:28—human IgG2 hinge); ELKTPLGDTTHTCPRCP (SEQ ID NO:29—human IgG3 hinge); SPNMVPHAHHAQ (SEQ ID NO:30); ESKYGPPCPPCP (SEQ ID NO:14) or ESKYGPPCPSCP (SEQ ID NO:32) (human IgG4 hinge-based) and the like. In some embodiments, the hinge region comprises SEQ ID NO:15. In some embodiments, the hinge region comprises SEQ ID NO:16.

The extracellular spacer can comprise an amino acid sequence of a human IgG, IgG2, IgG3, or IgG4, hinge region. The extracellular spacer may also include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:33).

The extracellular spacer can comprise an amino acid sequence derived from human CD8; e.g., the hinge region can comprise the amino acid sequence: TTTTPAPRPPTPAPTIASQPLSLRPEACRPAAGGAVHTRGLDFACD (SEQ LD NO:34), or a variant thereof.

The extracellular spacer may comprise or further comprise a CH2 region. An exemplary CH2 region is APEFEGGPSVFLFPPKPKDTLMISRTPEVTCVVVDVSQEDPEVQFNWYVDGVEVHNAKT KPREEQFQSTYRVVSVLTVLHQDWLNGKEYKCKVSNKGLPSSIEKTISKAK (SEQ ID NO:35). The extracellular spacer may comprise or further comprise a C H3 region. An exemplary CH3 region is

(SEQ ID NO: 36)
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLGK.

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some embodiments, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

D. Transmembrane Domain

Polypeptides of the present disclosure may comprise a transmembrane domain. In some embodiments, a transmembrane domain is a hydrophobic alpha helix that spans the membrane. Different transmembrane domains may result in different receptor stability.

In some embodiments, the transmembrane domain is interposed between the extracellular spacer and the cytoplasmic region. In some embodiments, the transmembrane domain is interposed between the extracellular spacer and one or more costimulatory regions. In some embodiments, a linker is between the transmembrane domain and the one or more costimulatory regions.

Any transmembrane domain that provides for insertion of a polypeptide into the cell membrane of a eukaryotic (e.g., mammalian) cell may be suitable for use. As one non-limiting example, the transmembrane sequence FWVLVVVGGVLACYSLLVTVAFIFWV (SEQ ID NO:17), which is CD28-derived, can be used. In some embodiments, the transmembrane domain is CD8 beta derived: LGLLVAGVLVLLVSLGVAIIHLCC (SEQ ID NO:37); CD4 derived: ALIVLGGVAGLLLFIGLGIFFCVRC (SEQ ID NO:38); CD3 zeta derived: LCYLLDGILFIYGVILTALFLRV (SEQ ID NO:39); CD28 derived: WVLVVVGGVLACYSLLVTVAFIIFWV (SEQ ID NO:40); CD134 (OX40) derived: VAAILGLGLVLGLLGPLAILLALYLL (SEQ ID NO:41); or CD7 derived: ALPAALAVISFLLGLGLGVACVLA (SEQ ID NO:42). In some embodiments, the transmembrane domain is derived from CD28, CD8, CD4, CD3-zeta, CD134, or CD7.

E. Cytoplasmic Region

After antigen recognition, receptors of the present disclosure may cluster and a signal transmitted to the cell through the cytoplasmic region. In some embodiments, the costimulatory domains described herein are part of the cytoplasmic region. In some embodiments, the cytoplasmic region comprises an intracellular signaling domain. An intracellular signaling domain may comprise a primary signaling domain and one or more costimulatory domains.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the cytoplasmic region includes at least one (e.g., one, two, three, four, five, six, etc.) ITAM motif as described herein. In some embodiments, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/1)), where n is an integer from 6 to 8, and each of the 6-8 X3 can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM n motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD33 (CD3 gamma); CD3-zeta; and CD79A (antigen receptor complex-associated protein alpha chain).

In some cases, the cytoplasmic region is derived from DAP12 (also known as TYROBP; TYRO protein tyrosine kinase binding protein; KARAP; PLOSL; DN AX-activation protein 12; KAR-associated protein; TYRO protein tyrosine kinase-binding protein; killer activating receptor associated protein; killer-activating receptor-associated protein; etc.). For example, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 43)
MGGLEPCSRLLLLPLLLAVSGLRPVQAQAQSDCSCSTVSPGVLAGIVMGD
LVLTVLIALAVYFLGRLVPRGRGAAEAATRKQRITETESPYQELQGQRSD
VYSDLNTQRPYYK;
(SEQ ID NO: 44)
MGGLEPCSRLLLLPLLLAVSGLRPVQAQAQSDCSCSTVSPGVLAGIVMGD
LVLTVLIALAVYFLGRLVPRGRGAAEATRKQRITETESPYQELQGQRSDV
YSDLNTQRPYYK;
(SEQ ID NO: 45)
MGGLEPCSRLLLLPLLLAVSDCSCSTVSPGVLAGIVMGDLVLTVLIALAV
YFLGRLVPRGRGAAEAATRKQRITETESPYQELQGQRSDVYSDLNTQRPY
YK;
or
(SEQ ID NO: 46)
MGGLEPCSRLLLLPLLLAVSDCSCSTVSPGVLAGIVMGDLVLTVLIALAV
YFLGRLVPRGRGAAEATRKQRITETESPYQELQGQRSDVYSDLNTQRPYY
K.

In some embodiments, a suitable cytoplasmic region can comprise an ITAM motif-containing portion of the full length DAP12 amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 47)
ESPYQELQGQRSDVYSDLNTQ.

In some embodiments, the cytoplasmic region is derived from FCER1G (also known as FCRG; Fe epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.). For example, a suitable endodomain polypeptide can comprise an amino acid sequence having at least 75%, at least 80%, at least 85%, at least 90%, at least 95%, at least 98%, or 100% amino acid sequence identity to

(SEQ ID NO: 48)
MIPAVVLLLLLLVEQAAALGEPQLCYILDAILFLYGIVLTLLYCRLKIQV
RKAAITSYEKSDGVYTGLSTRNQETYETLKIIEKPPQ.

In some embodiments, a suitable cytoplasmic region can comprise an ITAM motif-containing portion of the full length FCER1G amino acid sequence. Thus, a suitable endodomain polypeptide can comprise an amino acid sequence having at least 75%, at least 80%, at least 85%, at least 90%, at least 95%, at least 98%, or 100%, amino acid sequence identity to DGVYTGLSTRNQETYETLKHE (SEQ ID NO:49).

In some embodiments, the cytoplasmic region is derived from T cell surface glycoprotein CD3 delta chain (also known as CD3D; CD3-DELTA; T3D; CD3 antigen, delta subunit; CD3 delta; CD3δ; CD3d antigen, delta polypeptide (TiT3 complex); OKT3, delta chain; T cell receptor T3 delta chain; T cell surface glycoprotein CD3 delta chain; etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 170 aa, of either of the following amino acid sequences (2 isoforms):

(SEQ ID NO: 50)
MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQVYQ
PLRDRDDAQYSHLGGNWARNK
or
(SEQ ID NO: 51)
MEHSTFLSGLVLATLLSQVSPFKIPIEELEDRVFVNCNTSITWVEGTVGT
LLSDITRLDLGKRILDPRGIYRCNGTDIYKDKESTVQVHYRTADTQALLR
NDQVYQPLRDRDDAQYSHLGGNWARNK.

In some embodiments, a suitable cytoplasmic region can comprise an ITAM motif-containing portion of the full length CD3 delta amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 52)
DQVYQPLRDRDDAQYSHLGGN.

In some embodiments, the cytoplasmic region is derived from T cell surface glycoprotein CD3 epsilon chain (also known as CD3e, CD3ε; T cell surface antigen T3/Leu-4 epsilon chain, T cell surface glycoprotein CD3 epsilon chain, AI504783, CD3, CD3epsilon, T3e, etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 205 aa, of the following amino acid sequence:

(SEQ ID NO: 53)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI.

In some embodiments, a suitable cytoplasmic region can comprise an ITAM motif-containing portion of the full length CD3 epsilon amino acid sequence. Thus, a suitable endodomain polypeptide can comprise an amino acid sequence having at least 75%, at least 80%, at least 85%, at least 90%, at least 95%, at least 98%, or 100%, amino acid sequence identity to NPDYEPIRKGQRDLYSGLNQR (SEQ ID NO:54).

In some embodiments, the cytoplasmic region is derived from T cell surface glycoprotein CD3 gamma chain (also known as CD3G, CD3γ, T cell receptor T3 gamma chain, CD3-GAMMA, T3G, gamma polypeptide (TiT3 complex), etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 180 aa, of the following amino acid sequence:

(SEQ ID NO: 55)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN.

In some embodiments, a suitable cytoplasmic region can comprise an ITAM motif-containing portion of the full length CD3 gamma amino acid sequence. Thus, a suitable cytoplasmic region can comprise an amino acid sequence having at least 75%, at least 80%, at least 85%, at least 90%, at least 95%, at least 98%, or 100%, amino acid sequence identity to DQLYQPLKDREDQYSHLQGN (SEQ ID) NO:56).

In some embodiments, the cytopiasmic region is derived from T cell surface glycoprotein CD3 zeta chain (also known as CD3Z, CD3ζ, T cell receptor T3 zeta chain, CD247, CD3-ZETA, CD3H, CD3Q, T3Z, TCRZ, etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of th following amino acid sequences (2 isoforms):

(SEQ ID NO: 57)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
or
(SEQ ID NO: 58)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR.

In some embodiments, the cytoplasmic region comprises

(SEQ ID NO: 19)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

In some embodiments, a suitable cytoplasmic region can comprise an ITAM motif-containing portion of the full length CD3 zeta amino acid sequence. Thus, a suitable cytoplasmic region can comprise an amino acid sequence having at least 75%, at least 80%, at least 85%, at least 90%, at least 95%, at least 98%, or 100%, amino acid sequence identity to any of the following amino acid sequences:

(SEQ ID NO: 19)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR;
(SEQ ID NO: 61)
NQLYNELNLGRREEYDVLDKR;
(SEQ ID NO: 62)
EGLYNELQKDKMAEAYSEIGMK;
or
(SEQ ID NO: 63)
DGLYQGLSTATKDTYDALHMQ.

In some embodiments, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 150 aa, from about 150 aa to about 200 aa, or from about 200 aa to about 220 aa, of either of the following amino acid sequences (2 isoforms):

(SEQ ID NO: 64)
MPGGPGVLQALPATIFLLFLLSAVYLGPGCQALWMHKVPASLMVSLGED
AHFQCPHNSSNNANVTWWRVLHGNYTWPPEFLGPGEDPNGTLIIQNVNK
SHGGIYVCRVQEGNESYQQSCGTYLRVRQPPPRPFLDMGEGTKNRIITA
EGIILLFCAVVPGTLLLFRKRWQNEKLGLDAGDEYEDENLYEGLNLDDC
SMYEDISRGLQGTYQDVGSLNIGDVQLEKP;
or
(SEQ ID NO: 65)
MPGGPGVLQALPATIFLLFLLSAVYLGPGCQALWMHKVPASLMVSLGED
AHFQCPHNSSNNANVTWWRVLHGNYTWPPEFLGPGEDPNEPPPRPFLDM
GEGTKNRIITAEGIILLFCAVVPGTLLLFRKRWQNEKLGLDAGDEYEDE
NLYEGLNLDDCSMYEDISRGLQGTYQDVGSLNIGDVQLEKP.

In some embodiments, a suitable cytoplasmic region can comprise an ITAM motif-containing portion of the full length CD79A amino acid sequence. Thus, a suitable cytoplasmic region can comprise an amino acid sequence having at least 75%, at least 80%, at least 85%, at least 90%, at least 95%, at least 98%, or 100%, amino acid sequence identity to the following amino acid sequence: ENLYEGLNLDDCSMYEDISRG (SEQ ID NO:66).

In some embodiments, suitable cytoplasmic regions can comprise a DAP10/CD28 type signaling chain. An example of a CD28 signaling chain is the amino acid sequence FWVLVVVGGVLACYSLLVTVAFIIFWVRSKRSRLLHSDYMNMTPRRPGPTRKHYQPYA PPRDFAAYRS (SEQ ID NO:67). In some embodiments, a suitable endodomain comprises an amino acid sequence having at least 85%, at least 90%, at least 95%, at least 98%, or at least 99%, amino acid sequence identity to the entire length of the amino acid sequence

(SEQ ID NO: 67)
FWVLVVVGGVLACYSLLVTVAFHFWVRSKRSRLLHSDYMNMTPRRPGPTR
KHYQPYAPPRDFAAYRS.

Further cytoplasmic regions suitable for use in the polypeptides of the disclosure include a ZAP70 polypeptide, e.g., a polypeptide comprising an amino acid sequence having at least 85%, at least 90%, at least 95%, at least 98%, at least 99%, or 100%, amino acid sequence identity to a contiguous stretch of from about 300 amino acids to about 400 amino acids, from about 400 amino acids to about 500 amino acids, or from about 500 amino acids to 619 amino acids, of the following amino acid sequence:

(SEQ ID NO: 69)
MPDPAAHLPFFYGSISRAEAEEHLKLAGMADGLFLLRQCLRSLGGYVLS
LVHDVRFHHFPIERQLNGTYAIAGGKAHCGPAELCEFYSRDPDGLPCNL
RKPCNRPSGLEPQPGVFDCLRDAMVRDYVRQTWKLEGEALEQAIISQAP
QVEKLIATTAHERMPWYHSSLTREEAERKLYSGAQTDGKFLLRPRKEQG
TYALSLIYGKTVYHYLISQDKAGKYCIPEGTKFDTLWQLVEYLKLKADG
LIYCLKEACPNSSASNASGAAAPTLPAHPSTLTHPQRRIDTLNSDGYTP
EPARITSPDKPRPMPMDTSVYESPYSDPEELKDKKLFLKRDNLLIADIE
LGCGNFGSVRQGVYRMRKKQIDVAIKVLKQGTEKADTEEMMREAQIMHQ
LDNPYIVRLIGVCQAEALMLVMEMAGGGPLHKFLVGKREEIPVSNVAEL
LHQVSMGMKYLEEKNFVHRDLAARNVLLVNRHYAKISDFGLSKALGADD
SYYTARSAGKWPLKWYAPECINFRKFSSRSDVWSYGVTMWEALSYGQKP
YKKMKGPEVMAFIEQGKRMECPPECPPELYALMSDCWIYKWEDRPDFLT
VEQRMRACYYSLASKVEGPPGSTQKAEAACA.

1. Costimulatory Region

Non-limiting examples of suitable costimulatory regions, such as those included in the cytoplasmic region, include, but are not limited to, polypeptides from 4-IBB (CD137), CD28, ICOS, OX-40, BTLA, CD27, CD30, GITR, and HVEM.

A costimulatory region may have a length of at least, at most, or exactly 20, 25, 30, 35, 40, 50, 60, 70, 80, 90, 100, 150, 200, or 300 amino acids or any range derivable therein. In some embodiments, the costimulatory region is derived from an intracellular portion of the transmembrane protein 4-1BB (also known as TNFRSF9; CD137; CDw137; ILA; etc.). For example, a suitable costimulatory region can comprise an amino acid sequence having at least 75%, at least 80%, at least 85%, at least 90%, at least 95%, at least 98%, or 100% amino acid sequence identity to KRGRKKLLYIFKQPFMRPVQTTQEEDGCSCRFPEEEEGGCEL (SEQ ID NO:70).

In some embodiments, the costimulatory region is derived from an intracellular portion of the transmembrane protein CD28 (also known as Tp44). For example, a suitable costimulatory region can comprise an amino acid sequence having at least 75%, at least 80%, at least 85%, at least 90%, at least 95%, at least 98%, or 100% amino acid sequence identity to FWVRSKRSRLLHSDYMNMTPRRPGPTRKHYQPYAPPRDFAAYRS (SEQ ID NO:71).

In some embodiments, the costimulatory region is derived from an intracellular portion of the transmembrane protein ICOS (also known as AILM, CD278, and CVID1). For example, a suitable costimulatory region can comprise an amino acid sequence having at least 75%, at least 80%, at least 85%, at least 90%, at least 95%, at least 98%, or 100% amino acid sequence identity to TKKKYSSSVHDPNGEYMFMRAVNTAKKSRLTDVTL (SEQ ID NO:72).

In some embodiments, the costimulatory region is derived from an intracellular portion of the transmembrane protein OX-40 (also known as TNFRSF4, RP5-902P8.3, ACT35, CD134, OX40, TXGP1L). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 73)
RRDQRLPPDAHKPPGGGSFRTPIQEEQADAHSTLAKI.

In some embodiments, the costimulatory region is derived from an intracellular portion of the transmembrane protein BTLA (also known as BTLA1 and CD272). For example, a suitable costimulatory region can comprise an amino acid sequence having at least 75%, at least 80%, at least 85%, at least 90%, at least 95%, at least 98%, or 100% amino acid sequence identity to

(SEQ ID NO: 74)
CCLRRHQGKQNELSDTAGREINLVDAHLKSEQTEASTRQNSQVLLSETG
IYDNDPDLCFRMQEGSEVYSNPCLEENKPGIVYASLNHSVIGPNSRLAR
NVKEAPTEYASICVRS.

In some embodiments, the costimulatory region is derived from an intracellular portion of the transmembrane protein CD27 (also known as S 152, T14, TNFRSF7, and Tp55). For example, a suitable costimulatory region can comprise an amino acid sequence having at least 75%, at least 80%, at least 85%, at least 90%, at least 95%, at least 98%, or 100% amino acid sequence identity to

(SEQ ID NO: 75)
HQRRKYRSNKGESPVEPAEPCRYSCPREEEGSTIPIQEDYRKPEPACSP.

In some embodiments, the costimulatory region is derived from an intracellular portion of the transmembrane protein CD30 (also known as TNFRSF8, DIS166E, and Ki-1). For example, a suitable costimulatory region can comprise an amino acid sequence having at least 75%, at least 80%, at least 85%, at least 90%, at least 95%, at least 98%, or 100% amino acid sequence identity to

(SEQ ID NO: 76)
RRACRKRIRQKLHLCYPVQTSQPKLELVDSRPRRSSTQLRSGASVTEPVA
EERGLMSQPLMETCHSVGAAYLESLPLQDASPAGGPSSPRDLPEPRVSTE
HTNNKIEKIYIMKADTVIVGTVKAELPEGRGLAGPAEPELEEELEADHTP
HYPEQETEPPLGSCSDVMLSVEEEGKEDPLPTAASGK.

In some embodiments, the costimulatory region is derived from an intracellular portion of the transmembrane protein GITR (also known as TNFRSF18, RP5-902P8.2, AITR, CD357, and GITR-D). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 77)
HTWQLRSQCMWPRETQLLLEVPPSTEDARSCQFPEEERGERSAEEKGRLG
DLWV.

In some embodiments, the costimulatory region derived from an intracellular portion of the transmembrane protein HVEM (also known as TNFRSF14, RP3-395M20.6, ATAR, CD270, HVEA, HVEM, LIGHTR, and TR2). For example, a suitable costimulatory region can comprise an amino acid sequence having at least 75%, at least 80%, at least 85%, at least 90%, at least 95%, at least 98%, or 100% amino acid sequence identity to

(SEQ ID NO: 78)
CVKRRKPRGDVVKVIVSVQRKRQEAEGEATVIEALQAPPDVTTVAVEETI
PSFTGRSPNH.

In some embodiments, the costimulatory domain comprises an amino acid sequence having at least 75%, at least 80%, at least 85%, at least 90%, at least 95%, at least 98%, or 100% amino acid sequence identity to

(SEQ ID NO: 18)
RSKRSRGGHSDYMNMTPRRPGPTRKHYQPYAPPRDFAAYRS.

In some embodiments, the costimulatory domain amino acid sequence having at least 75%, at least 80%, at least 85%, at least 90%, at least 95%, at least 98%, or 100% amino acid sequence identity to a signaling domain from a cytokine receptor.

F. Detection Peptides

In some embodiments, the polypeptides described herein may further comprise a detection peptide (also “tag”). Suitable detection peptides include hemagglutinin (HA; e.g., YPYDVPDYA (SEQ ID NO:79); FLAG (e.g., DYKDDDDK (SEQ ID NO:80); c-myc (e.g., EQKLISEEDL; SEQ ID NO:81), and the like. In some embodiments, a polypeptide described herein further comprises a CD-20 mimotope peptide (e.g., CPYSNPSLC (SEQ ID NO:89). In some embodiments, a polypeptide described herein comprises a tag sequence having at least 75%, at least 80%, at least 85%, at least 90%, at least 95%, at least 98%, or 100% amino acid sequence identity to SEQ ID NO:89. Other suitable detection peptides are known in the art.

G. Peptide Linkers

In some embodiments, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G)n, glycine-serine polymers (including, for example, (GS)n, (GSGGS)n, (G4S)n and (GGGS)n, where n is an integer of at least one. In some embodiments, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:82), GGSGG (SEQ ID NO:83), GSGSG (SEQ ID NO:84), GSGGG (SEQ ID NO:85), GGGSG (SEQ ID NO:86), GSSSG (SEQ 11) NO:87), and the like.

In further embodiments, the linker comprises (EAAAK)n, wherein n is an integer of at least one. In some embodiments, n is at least, at most, or exactly 1, 2, 3, 4, 5, 6, 7, 8, 9, or 10 (or any derivable range therein).

In some embodiments, the linker is a Whitlow linker. In some embodiments, the linker comprises an amino acid sequence having at least 75%, at least 80%, at least 85%, at least 90%, at least 95%, at least 98%, or 100% amino acid sequence identity to GSTSGSGKPGSGEGSTKG (SEQ ID NO:9).

H. Additional Modifications and Polypeptide Enhancements

Additionally, the polypeptides of the disclosure may be chemically modified. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861).

It is contemplated that a region or fragment of a polypeptide of the disclosure may have an amino acid sequence that has, has at least or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, or contiguous amino acid deletions with respect to any of SEQ ID NOs:1-89. Alternatively, a region or fragment of a polypeptide of the disclosure may have an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOs: 1-89. Moreover, in some embodiments, a region or fragment comprises an amino acid region of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275,276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 in any of SEQ ID NOs: 1-89 (where position 1 is at the N-terminus of the SEQ ID NO). The polypeptides of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any range derivable therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 600, or more contiguous amino acids, or any range derivable therein, of any of SEQ ID NOs:1-89.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244,245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 44.3, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOs:1-89 (or any derivable range therein).

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or Y portions of the coding region.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### III. Cells

Certain embodiments relate to cells comprising polypeptides or nucleic acids of the disclosure. In some embodiments the cell is an immune cell. In some embodiments, the cell is a T cell. “T cell” (also “T-cell”) includes all types of immune cells expressing CD3 including, but not limited to, T-helper cells, invariant natural killer T (iNKT) cells, cytotoxic T cells, and T-regulatory cells (Treg) gamma-delta T cells. The T cell may refer to a CD4+ or CD8+ T cell. The T cell may refer to a CD62L-enriched T cell. An immune cell may be a natural killer (NK) cell, a B cell, or any other cell of the immune system.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

Cells of the present disclosure may comprise one or more therapeutic polypeptides or polynucleotides. In some embodiments, disclosed is a cell comprising one or more CAR polypeptides. In some embodiments, a cell comprises a CAR polypeptide comprising a tumor antigen binding domain. In some embodiments, a cell comprises a CAR polypeptide comprising a TYRP-1 binding domain. Cells comprising a CAR polypeptide may, in certain embodiments, further comprise one or more additional therapeutic polypeptides and/or polynucleotides. Cells comprising a therapeutic polypeptide or polynucleotide of the present disclosure may further comprise one or more additional genetic modifications (e.g., genetic mutations, gene deletions, gene additions, etc.) which, in some embodiments, improve the efficacy or safety of a therapeutic cell. Certain non-limiting examples of such genetic modifications are described in: Puig-Saus and Ribas. Gene editing: towards the third generation of adoptive T cell transfer therapies. Immuno- Oncology Technology. 2019 Jun. 13; 1:19-26, incorporated by reference herein in its entirety.

### IV. Methods for Modifying Genomic DNA

In certain embodiments, the genomic DNA is modified either to include additional mutations, insertions, or deletions, or to integrate certain molecular constructs of the disclosure so that the constructs are expressed from the genomic DNA. In some embodiments, a nucleic acid encoding a polypeptide of the disclosure is integrated into the genomic DNA of a cell. In some embodiments, a nucleic acid is integrated into a cell via viral transduction, such as gene transfer by lentiviral or retroviral transduction. In some embodiments, genomic DNA is modified by integration of nucleic acid encoding a polypeptide of the present disclosure (e.g., a CAR) into the genome of a host cell via a retroviral vector, a lentiviral vector, or an adeno-associated viral vector.

In some embodiments, the integration is targeted integration. In some embodiments, targeted integration is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids. One specific target is the TRAC (T cell receptor alpha constant) locus. For instance, cells would first be electroporated with a ribonucleoprotein (RNP) complex consisting of Cas9 protein complexed with a single-guide RNA (sgRNA) targeting the TRAC (T cell receptor alpha constant) locus. Fifteen minutes post electroporation, the cells would be treated with AAV6 carrying the homology directed repair (I-IDR) template that encodes for the CAR. In another example, double stranded or single stranded DNA comprises the HDR template and is introduced into the cell via electroporation together with the RNP complex.

Therefore, one aspect, the current disclosure includes targeted integration. One way of achieving this is through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, ΦC31 integrase, Bxb1-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRISPR/Cas-like endonucleases, I-Tevl nucleases or related monomeric hybrids, or artificial targeted DNA double strand break inducing agents. Exemplary targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases.

Multiple recognition sequences may be present in a single landing pad, allowing the landing pad to be targeted sequentially by two or more polynucleotide modification enzymes such that two or more unique nucleic acids (comprising, among other things, receptor genes and/or inducible reporters) can be inserted. Alternatively, the presence of multiple recognition sequences in the landing pad, allows multiple copies of the same nucleic acid to be inserted into the landing pad. When two nucleic acids are targeted to a single landing pad, the landing pad includes a first recognition sequence for a first polynucleotide modification enzyme (such as a first ZFN pair), and a second recognition sequence for a second polynucleotide modification enzyme (such as a second ZFN pair). Alternatively, or additionally, individual landing pads comprising one or more recognition sequences may be integrated at multiple locations. Increased protein expression may be observed in cells transformed with multiple copies of a payload. Alternatively, multiple gene products may be expressed simultaneously when multiple unique nucleic acid sequences comprising different expression cassettes are inserted, whether in the same or a different landing pad. Regardless of the number and type of nucleic acid, when the targeting endonuclease is a ZFN, exemplary ZFN pairs include hSIRT, hRSK4, and hAAVS1, with accompanying recognition sequences.

Generally speaking, a landing pad used to facilitate targeted integration may comprise at least one recognition sequence. For example, a landing pad may comprise at least one, at least two, at least three, at least four, at least five, at least six, at least seven, at least eight, at least nine, or at least ten or more recognition sequences. In embodiments comprising more than one recognition sequence, the recognition sequences may be unique from one another (i.e. recognized by different polynucleotide modification enzymes), the same repeated sequence, or a combination of repeated and unique sequences.

One of ordinary skill in the art will readily understand that an exogenous nucleic acid used as a landing pad may also include other sequences in addition to the recognition sequence(s). For example, it may be expedient to include one or more sequences encoding selectable or screenable genes as described herein, such as antibiotic resistance genes, metabolic selection markers, or fluorescence proteins. Use of other supplemental sequences such as transcription regulatory and control elements (i.e., promoters, partial promoters, promoter traps, start codons, enhancers, introns, insulators and other expression elements) can also be present.

In addition to selection of an appropriate recognition sequence(s), selection of a targeting endonuclease with a high cutting efficiency also improves the rate of targeted integration of the landing pad(s). Cutting efficiency of targeting endonucleases can be determined using methods well-known in the art including, for example, using assays such as a CEL-1 assay or direct sequencing of insertions/deletions (Indels) in PCR amplicons.

The type of targeting endonuclease used in the methods and cells disclosed herein can and will vary. The targeting endonuclease may be a naturally-occurring protein or an engineered protein. One example of a targeting endonuclease is a zinc-finger nuclease, which is discussed in further detail below.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named “LAGLIDADG” has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31(11):2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7(1):171-192; Bogdanove A J, Voytas D F., 2011, Science, 333(6051):1843-6; Bradley P, Bogdanove A J, Stoddard B L., 2013, Curr Opin Struct Biol., 23(1):93-9.

Another exemplary targeting endonuclease is a site-specific nuclease. In particular, the site-specific nuclease may be a “rare-cutter” endonuclease whose recognition sequence occurs rarely in a genome. Preferably, the recognition sequence of the site-specific nuclease occurs only once in a genome. Alternatively, the targeting nuclease may be an artificial targeted DNA double strand break inducing agent.

In some embodiments, targeted integrated can be achieved through the use of an integrase. For example, The phiC31 integrase is a sequence-specific recombinase encoded within the genome of the bacteriophage phiC31. The phiC31 integrase mediates recombination between two 34 base pair sequences termed attachment sites (att), one found in the phage and the other in the bacterial host. This serine integrase has been show to function efficiently in many different cell types including mammalian cells. In the presence of phiC31 integrase, an attB-containing donor plasmid can be unidirectional integrated into a target genome through recombination at sites with sequence similarity to the native attP site (termed pseudo-attP sites). phiC31 integrase can integrate a plasmid of any size, as a single copy, and requires no cofactors. The integrated transgenes are stably expressed and heritable.

In one embodiment, genomic integration of polynucleotides of the disclosure is achieved through the use of a transposase. For example, a synthetic DNA transposon (e.g. “Sleeping Beauty” transposon system) designed to introduce precisely defined DNA sequences into the chromosome of vertebrate animals can be used. The Sleeping Beauty transposon system is composed of a Sleeping Beauty (SB) transposase and a transposon that was designed to insert specific sequences of DNA into genomes of vertebrate animals. DNA transposons translocate from one DNA site to another in a simple, cut-and-paste manner. Transposition is a precise process in which a defined DNA segment is excised from one DNA molecule and moved to another site in the same or different DNA molecule or genome.

As do all other Tc1/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, in which case the transposon becomes a non-autonomous element. Non-autonomous transposons are most useful as genetic tools because after insertion they cannot independently continue to excise and re-insert. All of the DNA transposons identified in the human genome and other mammalian genomes are non-autonomous because even though they contain transposase genes, the genes are non-functional and unable to generate a transposase that can mobilize the transposon.

### V. Methods

Aspects of the current disclosure relate to methods for treating cancer, such as skin cancer. In some embodiments, disclosed are methods for treating a subject having melanoma. In some embodiments, disclosed are methods for treating a TYRP-1⁺ cancer. In further embodiments, the therapeutic receptors (e.g., CARs) described herein may be used for stimulating an immune response. The immune response stimulation may be done in vitro, in vivo, or ex vivo. In some embodiments, the therapeutic receptors described herein are for preventing relapse. The method generally involves genetically modifying a mammalian cell with an expression vector, or a DNA, an RNA (e.g., in vitro transcribed RNA), or an adeno-associated virus (AAV) comprising nucleotide sequences encoding a polypeptide of the disclosure or directly transferring the polypeptide to the cell. The cell can be an immune cell (e.g., a T lymphocyte or NK cell), a stem cell, a progenitor cell, etc. In some embodiments, the cell is a cell described herein.

In some embodiments, the genetic modification is carried out ex vivo. For example, a T lymphocyte, a stem cell, or an NK cell (or cell described herein) is obtained from an individual; and the cell obtained from the individual is genetically modified to express a polypeptide of the disclosure. In some cases, the genetically modified cell is activated ex vivo. In other cases, the genetically modified cell is introduced into an individual (e.g., the individual from whom the cell was obtained); and the genetically modified cell is activated in vivo.

In some embodiments, the methods relate to administration of the cells or peptides described herein for the treatment of a cancer or administration to a person with a cancer. In some embodiments, the cancer is a TYRP-1⁺ cancer. In some embodiments, the cancer is skin cancer. In some embodiments, the cancer is melanoma. In some embodiments, the melanoma is superficial spreading melanoma, modular melanoma, acral-lentiginous melanoma, lentigo maligna melanoma, amelanotic melanoma, desmoplastic melanoma, ocular melanoma, mucosal melanoma, or metastatic melanoma. In further embodiments, a treatment with respect to cancer discussed herein may apply to precancer, such as actinic keratosis (AK). In other embodiments, a cancer can be squamous cell carcinoma of the skin. In additional embodiments, a patient previously had a melanoma or precancerous melanoma and/or is at risk for melanoma.

### VI. Additional Therapies

A. Immunotherapy

In some embodiments, the methods comprise administration of a cancer immunotherapy. Cancer immunotherapy (sometimes called immuno-oncology, abbreviated 10) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

1. Checkpoint Inhibitors and Combination Treatment

Embodiments of the disclosure may include administration of immune checkpoint inhibitors (also referred to as checkpoint inhibitor therapy), which are further described below. The checkpoint inhibitor therapy may be a monotherapy, targeting only one cellular checkpoint proteins or may be combination therapy that targets at least two cellular checkpoint proteins. For example, the checkpoint inhibitor monotherapy may comprise one of: a PD-1, PD-L1, or PD-L2 inhibitor or may comprise one of a CTLA-4, B7-1, or B7-2 inhibitor. The checkpoint inhibitor combination therapy may comprise one of: a PD-1, PD-L1, or PD-L2 inhibitor and, in combination, may further comprise one of a CTLA-4, B7-1, or B7-2 inhibitor. The combination of inhibitors in combination therapy need not be in the same composition, but can be administered either at the same time, at substantially the same time, or in a dosing regimen that includes periodic administration of both of the inhibitors, wherein the period may be a time period described herein.

a. PD-1, PD-L1, and PD-L2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect, the PD-1 ligand binding partners are PD-L1 and/or PD-L2. In another embodiment, a PD-L1 inhibitor is a molecule that inhibits the binding of PD-L1 to its binding partners. In a specific aspect, PD-L1 binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. patent application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-L1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PD-L1, or PD-L2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2 Inhibitors

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment, they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

5. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Tumor targeted T cells can be generated through gene therapy. Tumor targeted T cells can be expanded by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

B. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

C. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

D. Neoantigens

In some embodiments, the additional therapy comprises targeting of neoantigen mutations. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

E. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

F. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VII. Pharmaceutical Compositions

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising cells expressing a therapeutic receptor) are administered via intravenous injection.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 M to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 M to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VIII. Sequences

The amino acid sequence of example chimeric polypeptides and CAR molecules useful in the methods and compositions of the present disclosure are provided in Table 1 below.

TABLE 1
CARs

| | SEQ | |
|---|---|---|
| Name | ID NO: | Sequence |
| 20D7SS | 1 | METDTLLLWVLLLWVPGSTGEIVLTQSPATLSLSP |
| | | GERATLSCRASQSVSSYLAWYQQKPGQAPRLLIYD |
| | | ASNRATGIPARFSGSGSGTDFTLTISSLEPEDFAV |
| | | YYCQQRSNWLMYTFGQGTKLEIKGSTSGSGKPGSG |
| | | EGSTKGQVQLVQSGSELKKPGASVKISCKASGYTF |
| | | TSYAMNWVRQAPGQGLESMGWINTNTGNPTYAQGF |
| | | TGRFVFSMDTSVSTAYLQISSLKAEDTAIYYCAPR |
| | | YSSSWYLDYWGQGTLVTVSSESKYGPPCPPCPFWV |
| | | LVVVGGVLACYSLLVTVAFIIFWVRSKRSRGGHSD |
| | | YMNMTPRRPGPTRKHYQPYAPPRDFAAYRSRVKFS |
| | | RSADAPAYQQGQNQLYNELNLGRREEYDVLDKRRG |
| | | RDPEMGGKPRRKNPQEGLYNELQKDKMAEAYSEIG |
| | | MKGERRRGKGHDGLYQGLSTATKDTYDALHMQALP |
| | | PR |
| 20D7SM | 2 | METDTLLLWVLLLWVPGSTGEIVLTQSPATLSLSP |
| | | GERATLSCRASQSVSSYLAWYQQKPGQAPRLLIYD |
| | | ASNRATGIPARFSGSGSGTDFTLTISSLEPEDFAV |
| | | YYCQQRSNWLMYTFGQGTKLEIKGSTSGSGKPGSG |
| | | EGSTKGQVQLVQSGSELKKPGASVKISCKASGYTF |
| | | TSYAMNWVRQAPGQGLESMGWINTNTGNPTYAQGF |
| | | TGRFVFSMDTSVSTAYLQISSLKAEDTAIYYCAPR |
| | | YSSSWYLDYWGQGTLVTVSSESKYGPPCPPCPGQP |
| | | REPQVYTLPPSQEEMTKNQVSLTCLVKGFYPSDIA |
| | | VEWESNGQPENNYKTTPPVLDSDGSFFLYSRLTVD |
| | | KSRWQEGNVFSCSVMHEALHNHYTQKSLSLSLGKF |
| | | WVLVVVGGVLACYSLLVTVAFIIFWVRSKRSRGGH |
| | | SDYMNMTPRRPGPTRKHYQPYAPPRDFAAYRSRVK |
| | | FSRSADAPAYQQGQNQLYNELNLGRREEYDVLDKR |
| | | RGRDPEMGGKPRRKNPQEGLYNELQKDKMAEAYSE |
| | | IGMKGERRRGKGHDGLYQGLSTATKDTYDALHMQA |
| | | LPPR |
| 20D7SL | 3 | METDTLLLWVLLLWVPGSTGEIVLTQSPATLSLSP |
| (also | | GERATLSCRASQSVSSYLAWYQQKPGQAPRLLIYD |
| “20D7SL- | | ASNRATGIPARFSGSGSGTDFTLTISSLEPEDFAV |
| 28z”) | | YYCQQRSNWLMYTFGQGTKLEIKGSTSGSGKPGSG |
| | | EGSTKGQVQLVQSGSELKKPGASVKISCKASGYTF |
| | | TSYAMNWVRQAPGQGLESMGWINTNTGNPTYAQGF |
| | | TGRFVFSMDTSVSTAYLQISSLKAEDTAIYYCAPR |
| | | YSSSWYLDYWGQGTLVTVSSESKYGPPCPPCPAPE |
| | | FEGGPSVFLFPPKPKDTLMISRTPEVTCVVVDVSQ |
| | | EDPEVQFNWYVDGVEVHNAKTKPREEQFQSTYRVV |
| | | SVLTVLHQDWLNGKEYKCKVSNKGLPSSIEKTISK |
| | | AKGQPREPQVYTLPPSQEEMTKNQVSLTCLVKGFY |
| | | PSDIAVEWESNGQPENNYKTTPPVLDSDGSFFLYS |
| | | RLTVDKSRWQEGNVFSCSVMHEALHNHYTQKSLSL |
| | | SLGKFWVLVVVGGVLACYSLLVTVAFIIFWVRSKR |
| | | SRGGHSDYMNMTPRRPGPTRKHYQPYAPPRDFAAY |
| | | RSRVKFSRSADAPAYQQGQNQLYNELNLGRREEYD |
| | | VLDKRRGRDPEMGGKPRRKNPQEGLYNELQKDKMA |
| | | EAYSEIGMKGERRRGKGHDGLYQGLSTATKDTYDA |
| | | LHMQALPPR |
| 20D7SL- | 88 | METDTLLLWVLLLWVPGSTGEIVLTQSPATLSLSP |
| BBZ | | GERATLSCRASQSVSSYLAWYQQKPGQAPRLLIYD |
| | | ASNRATGIPARFSGSGSGTDFTLTISSLEPEDFAV |
| | | YYCQQRSNWLMYTFGQGTKLEIKGSTSGSGKPGSG |
| | | EGSTKGQVQLVQSGSELKKPGASVKISCKASGYTF |
| | | TSYAMNWVRQAPGQGLESMGWINTNTGNPTYAQGF |
| | | TGRFVFSMDTSVSTAYLQISSLKAEDTAIYYCAPR |
| | | YSSSWYLDYWGQGTLVTVSSESKYGPPCPPCPAPE |
| | | FEGGPSVFLFPPKPKDTLMISRTPEVTCVVVDVSQ |
| | | EDPEVQFNWYVDGVEVHNAKTKPREEQFQSTYRVV |
| | | SVLTVLHQDWLNGKEYKCKVSNKGLPSSIEKTISK |
| | | AKGQPREPQVYTLPPSQEEMTKNQVSLTCLVKGFY |
| | | PSDIAVEWESNGQPENNYKTTPPVLDSDGSFFLYS |
| | | RLTVDKSRWQEGNVFSCSVMHEALHNHYTQKSLSL |
| | | SLGKFWVLVVVGGVLACYSLLVTVAFIIFWVKRGR |
| | | KKLLYIFKQPFMRPVQTTQEEDGCSCRFPEEEEGG |
| | | CELRVKFSRSADAPAYQQGQNQLYNELNLGRREEY |
| | | DVLDKRRGRDPEMGGKPRRKNPQEGLYNELQKDKM |
| | | AEAYSEIGMKGERRRGKGHDGLYQGLSTATKDTYD |
| | | ALHMQALPPR |

Example CDR embodiments of a TYRP-1 binding region of the present disclosure include those provided in Table 2 below.

TABLE 2
CDRs for TYRP-1 binding region

| Name | SEQ ID NO: | Sequence |
|---|---|---|
| LCDR1 | 6 | RASQSVSSYLA |
| LCDR2 | 7 | DASNRAT |
| LCDR3 | 8 | QQRSNWLMYT |
| HCDR1 | 11 | GYTFTSYAMN |
| HCDR2 | 12 | WINTNTGNPTYAQGFTG |
| HCDR3 | 13 | RYSSSWYLDY |

Additional polypeptides, domains, and regions useful in the methods and compositions of the present disclosure are provided in Table 3 below.

TABLE 3
Polypeptide domains useful in
embodiments of the disclosure

| | SEQ | |
|---|---|---|
| Name | ID NO: | Sequence |
| Murine Kappa Chain | 4 | METDTLLLWVLLLWVPGSTG |
| Signal Peptide | | |
| VL | 5 | EIVLTQSPATLSLSPGERATLSCR |
| | | ASQSVSSYLAWYQQKPGQAPRLLI |
| | | YDASNRATGIPARFSGSGSGTDFT |
| | | LTISSLEPEDFAVYYCQQRSNWLM |
| | | YTFGQGTKLEIK |
| Whitlow linker | 9 | GSTSGSGKPGSGEGSTKG |
| VH | 10 | QVQLVQSGSELKKPGASVKISCKA |
| | | SGYTFTSYAMNWVRQAPGQGLESM |
| | | GWINTNTGNPTYAQGFTGRFVFSM |
| | | DTSVSTAYLQISSLKAEDTAIYYC |
| | | APRYSSSWYLDYWGQGTLVTVSS |
| Hinge-Short | 14 | ESKYGPPCPPCP |
| Hinge-Medium | 15 | ESKYGPPCPPCPGQPREPQVYTLP |
| | | PSQEEMTKNQVSLTCLVKGFYPSD |
| | | IAVEWESNGQPENNYKTTPPVLDS |
| | | DGSFFLYSRLTVDKSRWQEGNVFS |
| | | CSVMHEALHNHYTQKSLSLSLGK |
| Hinge-Long | 16 | ESKYGPPCPPCPAPEFEGGPSVFL |
| | | FPPKPKDTLMISRTPEVTCVVVDV |
| | | SQEDPEVQFNWYVDGVEVHNAKTK |
| | | PREEQFQSTYRVVSVLTVLHQDWL |
| | | NGKEYKCKVSNKGLPSSIEKTISK |
| | | AKGQPREPQVYTLPPSQEEMTKNQ |
| | | VSLTCLVKGFYPSDIAVEWESNGQ |
| | | PENNYKTTPPVLDSDGSFFLYSRL |
| | | TVDKSRWQEGNVFSCSVMHEALHN |
| | | HYTQKSLSLSLGK |
| CD28 | 17 | FWVLVVVGGVLACYSLLVTVAFII |
| Transmembrane | | FWV |
| Domain | | |
| CD28 Costimulatory | 18 | RSKRSRGGHSDYMNMTPRRPGPTR |
| Domain | | KHYQPYAPPRDFAAYRS |
| CD3ζ Primary | 19 | RVKFSRSADAPAYQQGQNQLYNEL |
| Signaling Domain | | NLGRREEYDVLDKRRGRDPEMGGK |
| | | PRRKNPQEGLYNELQKDKMAEAYS |
| | | EIGMKGERRRGKGHDGLYQGLSTA |
| | | TKDTYDALHMQALPPR |

### IX. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. The Examples should not be construed as limiting in any way. The contents of all cited references (including literature references, issued patents, published patent applications, and GenBank Accession numbers as cited throughout this application) are hereby expressly incorporated by reference. When definitions of terms in documents that are incorporated by reference herein conflict with those used herein, the definitions used herein govern.

### Example 1: In Vitro Cytotoxicity in a Panel of Human Melanoma Cell Lines with Different Levels of TYRP-1 Expression

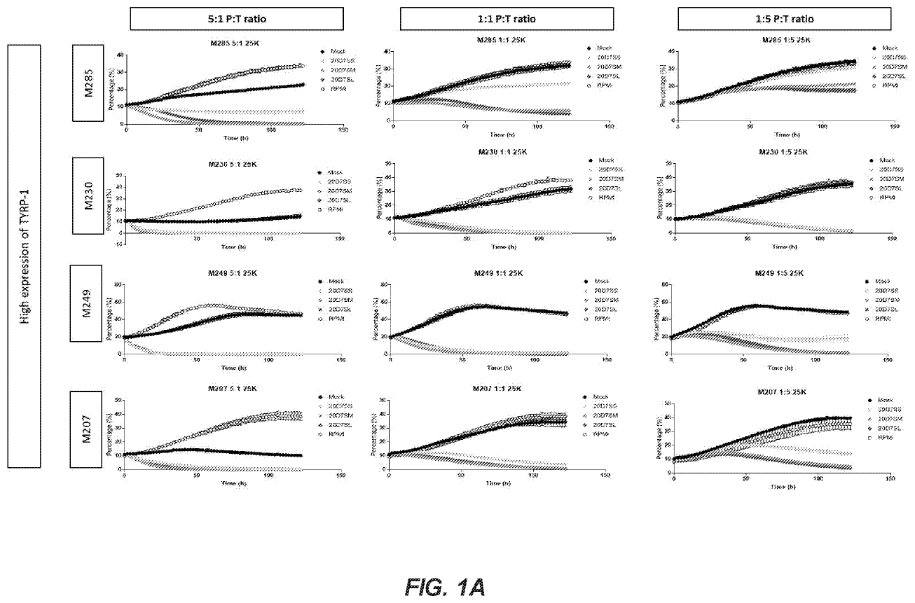

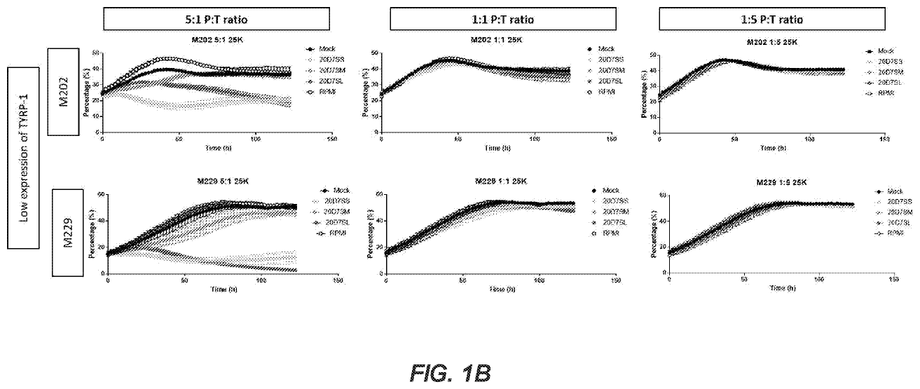

Human PBMCs were activated and transduced with a retroviral vector encoding for either 20D7SS (SEQ ID NO:1), 20D7SM (SEQ ID NO:2), or 20D7SL (SEQ ID NO:3) CAR constructs. T cells were expanded for 5 days and subsequently co-cultured with melanoma cell monolayers using a 5:1, 1:1, or 1:5 T cell product-to-melanoma cell (P:T) ratio. Untransduced T cells (mock) or melanoma cells cultured in media only (RPMI) were used as control. The melanoma cell lines used stably express nuclear RFP (nRFP). To measure cytotoxicity, the percentage of nRFP was followed over time. Results for melanoma cells with high TYRP-1 expression, M285, M230, M249, and M207, are shown in FIG. 1A. At least one of the tested CAR constructs was effective in reducing melanoma cell viability in all four cell lines at each of the ratios measured (5:1, 1:1, and 1:5). Results for melanoma cells with low TYRP-1 expression, M202 and M229, are shown in FIG. 1B. Mean±SD are shown in the graphs.

### Example 2: In Vitro Cytokine Secretion by T Cells Upon Co-Culture with a Panel of Human Melanoma Cell Lines with Different Levels of TYRP-1 Expression

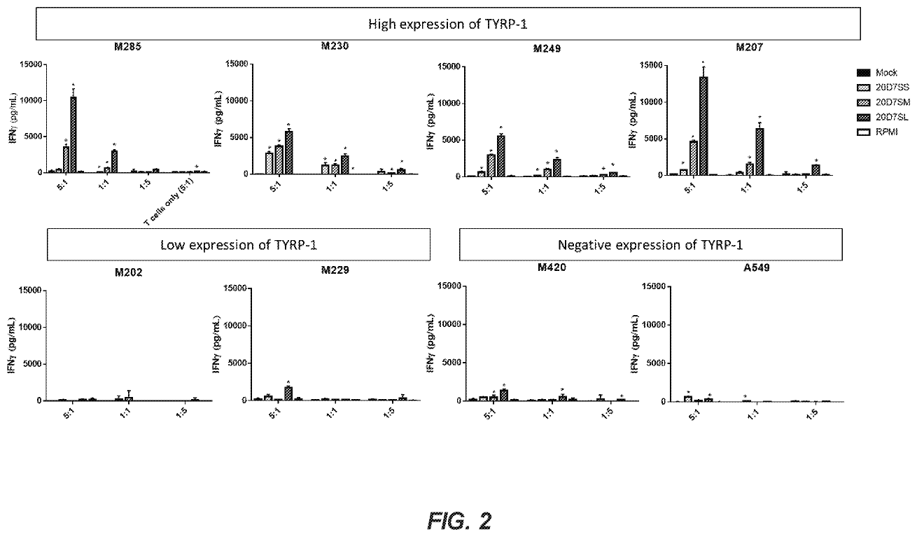

Human PBMCs were activated and transduced with a retroviral vector encoding for either 20D7SS (SEQ ID NO:1), 20D7SM (SEQ ID NO:2), or 20D7SL (SEQ ID NO:3) CAR constructs. T cells were expanded for 5 days and subsequently co-cultured with melanoma cell monolayers using a 5:1, 1:1, or 1:5 T cell product-to-melanoma cell (P:T) ratio. Untransduced T cells (mock) were used as control. Twenty-four hours after co-culture, the supernatants were collected and the IFNγ secretion was quantified by ELISA. As an additional control, the secretion of IFNγ was also measured in the absence of target melanoma cells (RPMI, T cell only control). The results for all cells are shown in FIG. 2. Mean±SD are shown in the graph. * indicates p<0.05 vs. mock T cells using a t test with Holm-Sidak correction for multiple comparison. For each ratio tested (5:1, 1:1, and 1:5), FIG. 2 shows, from left to right, IFNγ secretion from treatment with Mock cells, 20D7SS, 20D7SM, 20D7SL, and RPMI.

### Example 3: In Vitro Cytotoxicity and Cytokine Secretion in a Murine Melanoma Cell Line

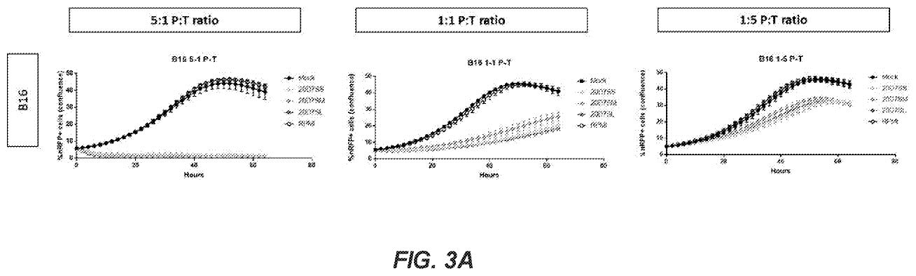

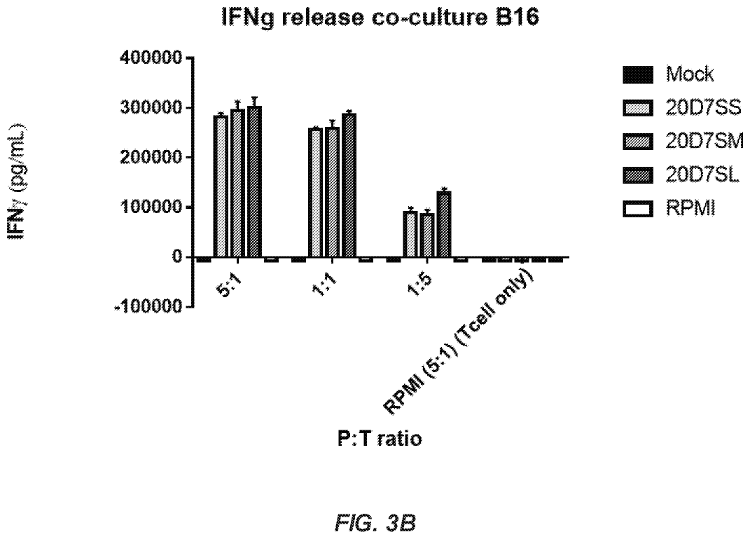

C57/B6 mice were euthanized, their spleens collected, and the CD3+ T cells purified and activated with CD3/28 beads and interleukin-2 (IL2). Twenty-four hours after activation, the T cells were transduced with retroviral vectors encoding for either 20D7SS (SEQ ID NO:1), 20D7SM (SEQ ID NO:2), or 20D7SL (SEQ ID NO:3) CAR constructs. The T cells were expanded for 6 days and subsequently co-cultured with B16-F10 melanoma monolayers using a 5:1, 1:1 or 1:5 T cell product-to-melanoma cell (P:T) ratio. Untransduced T cells (mock) or cell culture media only (RPMI) were used as control. The cytotoxicity of the T cells was measured by the percentage of nRFP confluence over time; these results are shown in FIG. 3A. Treatment with all three CAR constructs resulted in reduced cell viability, relative to Mock or RPMI treatment, at all three ratios (5:1, 1:1, and 1:5). The B16-F10 cell line was previously modified to constitutively express nRFP. 24 hours after co-culture, the presence of IFNγ was quantified by ELISA. As an additional control, the secretion of IFNγ was also measured in the absence of target melanoma cells (RPMI, T cell only control). These results are shown in FIG. 3B. Treatment with all three CAR constructs stimulated IFNγ at all three ratios (5:1, 1:1, and 1:5), while the control treatments did not. Mean±SD are shown in the graphs.

### Example 4: In Vivo Antitumor Activity of the 20D7SS, 20D7SM, and 20D7SL CAR Constructs in an Immunocompetent Murine Model

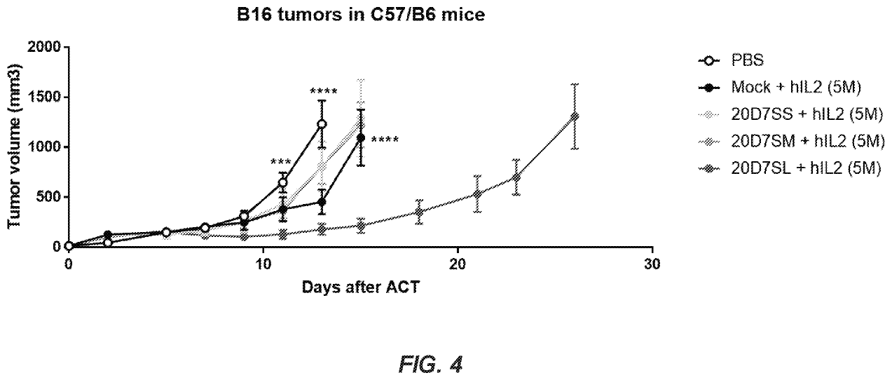

Murine T cells from C57/B6 mice were purified, transduced, and expanded as described in Example 3. Four days after transduction, 5 million T cells transduced with retroviral vectors encoding for either 20D7SS (SEQ ID NO:1), 20D7SM (SEQ ID NO:2), or 20D7SL (SEQ ID NO:3) CAR constructs were administered intravenously into C57/B6 mice bearing B16-F10 melanoma tumors. Untransduced T cells (mock) or PBS were used as control. Mice were preconditioned the day before T cell administration with lymphodepleting total body irradiation (500cGy). Three doses of 50,000 IU/mice of human IL2 were administered at day 0, 1 and 2 after T cell transfer. The tumor volumes were followed overtime using a caliper. The results are shown in FIG. 4. Mean±SD are shown in the graphs. *** p<0.001, ****p<0.0001 compared to 20D7SL CAR treated group using a two-way ANOVA with Tukey correction for multiple comparison. Treatment with cells expressing 20D7SL significantly delayed tumor growth in this model relative to control treatments.

### Example 5: In Vivo Antitumor Activity of the 20D7SL CAR Constructs in an Immunocompetent Murine Model

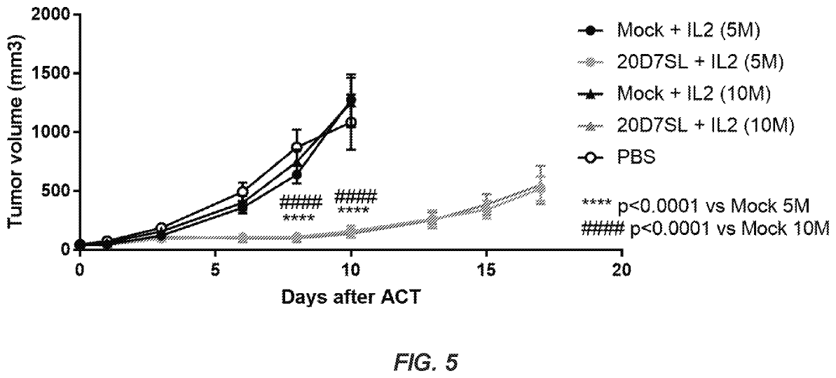

Murine T cells from C57/B6 mice were purified, transduced, and expanded as described in Example 3. Four days after transduction, 5 million (5M) or 10 million (10M) T cells transduced with the retroviral vectors encoding for the 20D7SL (SEQ ID NO:3) CAR construct were administered intravenously into C57/B6 mice bearing B16-F10 melanoma tumors. Untransduced T cells (mock) or PBS were used as control. Mice were preconditioned the day before T cell administration with lymphodepleting total body irradiation (500cGy). Three doses of 50,000 IU/mice of human IL2 were administered at day 0, 1 and 2 after T cell transfer. The tumor volumes were followed overtime using a caliper. The results are shown in FIG. 5. Mean±SD are shown in the graph. **** p<0.0001 compared to Mock 5M treated group, ####p<0.0001 compared to Mock 10M treated group using a two-way ANOVA with Tukey correction for multiple comparison. Treatment with both 5M and 10M cells expressing 20D7SL significantly delayed tumor growth in this model relative to control treatments.

### Example 6: In Vivo Antitumor Activity of the 20D7SL CAR Constructs Alone or in Combination with Standard IL-2 Treatment in an Immunocompetent Murine Model

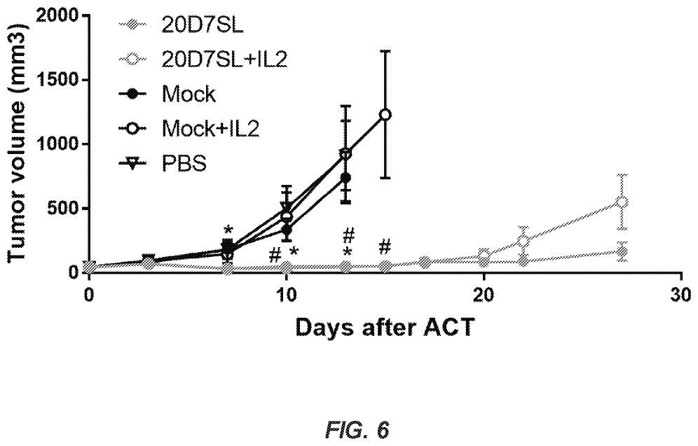

Murine T cells from C57/B6 mice were purified, activated, transduced, and expanded for five days. Four days after transduction, 10 million T cells transduced with a retroviral vector encoding for 20D7SL CAR construct were administered intravenously into C57/B6 mice bearing B16-F10 melanoma tumors. Untransduced T cells (mock) or PBS were used as control. Mice were preconditioned the day before T cell administration with lymphodepleting total body irradiation (500cGy). Three doses of 50,000 IU/mice of human IL-2 were administered at day 0, 1 and 2 after T cell transfer in the indicated groups. The tumor volumes were followed overtime using a caliper (mean±SD are shown in the graph). The results are shown in FIG. 6. * p<0.05 vs Mock; #p<0.05 vs Mock+IL2, unpaired t-test with Holm-Sidak correction for multiple comparison. Treatment with cells expressing 20D7SL, with and without 11-2, significantly delayed tumor growth in this model relative to control treatments.

### Example 7: In Vivo Antitumor Activity of the 20D7SL CAR T in Patient-Derived Melanoma Models in Immunodeficient Mouse Models

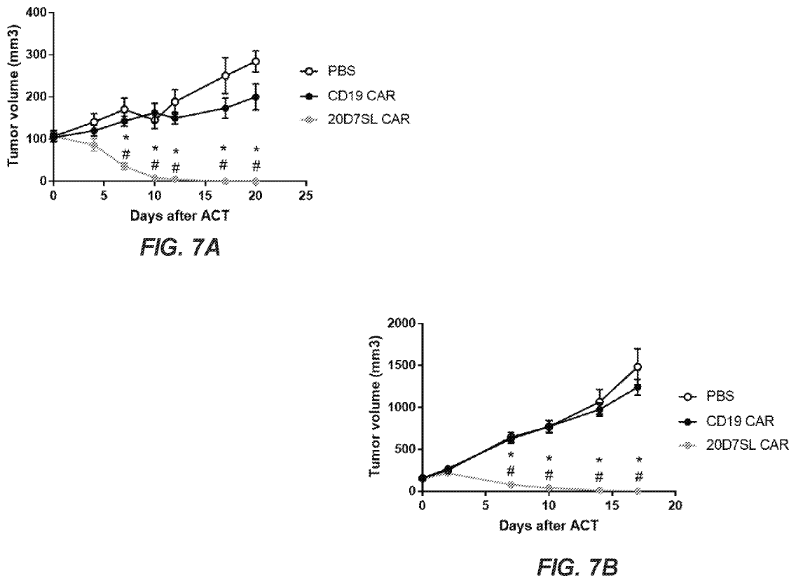

Human PBMCs were activated, transduced with a retroviral vector expressing the 20D7SL CAR and expanded for 9 days. 10 million T cells transduced with retroviral vectors encoding for 20D7SL CAR construct were administered intravenously into NSG mice bearing M207 (FIG. 7A) and M249 (FIG. 7B) subcutaneous tumors. CD19 CAR-T cells or vehicle only were used as a negative control. The tumor volumes were followed overtime using a caliper (mean±SD are shown in the graph). The results are shown in FIG. 7A (M207 cells) and FIG. 7B (M249 cells). * p<0.05 vs PBS; #p<0.05 vs CD19 CAR-T cells, unpaired t-test with Holm-Sidak correction for multiple comparison.

### Example 8: In Vitro Cytotoxicity Overtime of the 20D7SL CAR Constructs in a Panel of Human Non-Melanoma Cell Lines with Negative Expression of TYRP-1

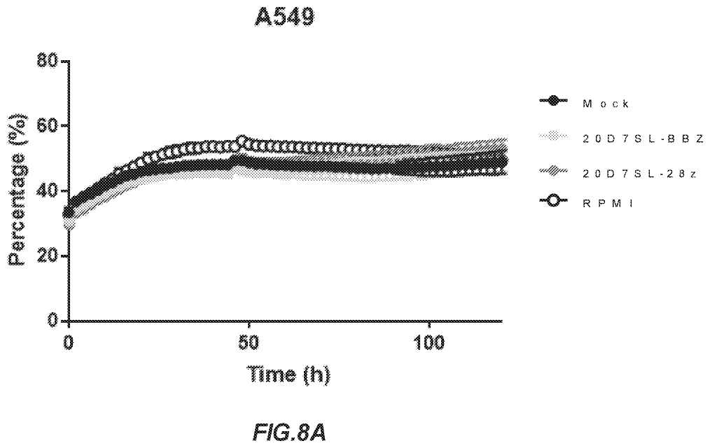

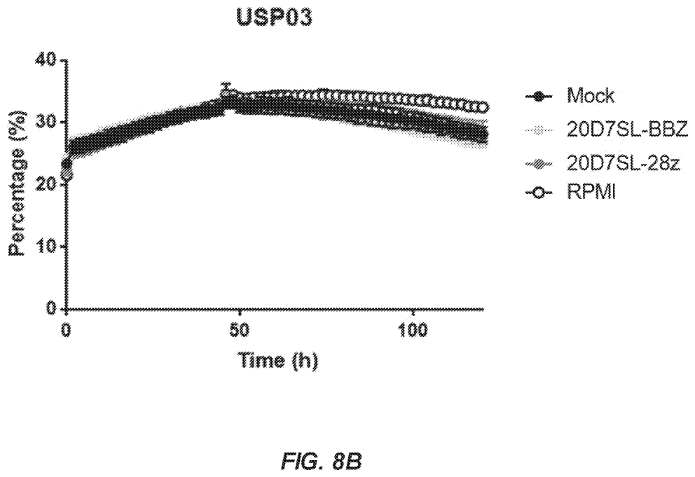

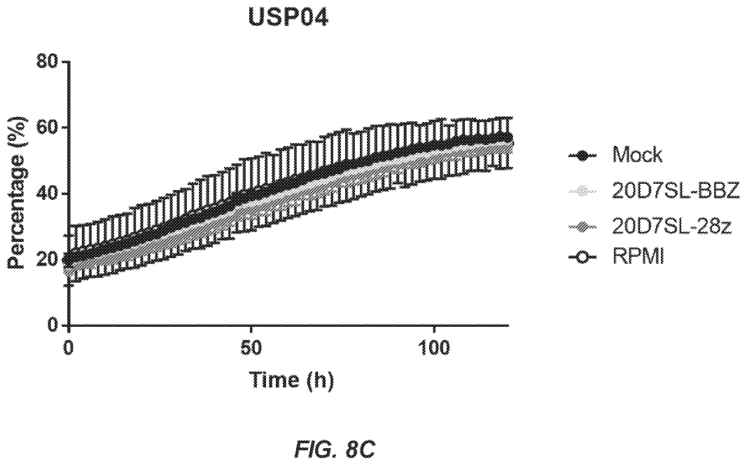

Human PBMCs were activated, transduced with lentiviral vectors encoding for either the 20D7SL-28z CAR construct (comprising a CD28 co-stimulatory signaling domain) or the 20D7SL-BBZ construct (comprising a 4-1BB co-stimulatory signaling domain). These cells were expanded for 9 days. CAR-T cells and controls were co-cultured with A549 (FIG. 8A, lung adenocarcinoma), UPS-03 (FIG. 8B, sarcoma), and UPS-04 (FIG. 8C, sarcoma) cells using a 1:1 T cell product-to-tumor cell ratio. Untransduced T cells (mock) or cell culture media only (RPMI) were used as control. These non-melanoma tumor cell lines stably express nuclear RFP (nRFP). To measure cytotoxicity, the percentage of nRFP was followed over time. The results are shown in FIGS. 8A-8C. Mean±SD are shown in the graph. Viability was not reduced in cell lines having negative expression of TYRP-1.

### Example 9: Loss of In Vitro Cytokine Secretion and Cytotoxicity Upon Co-Culture with TYRP-1 Knockout Cell Lines

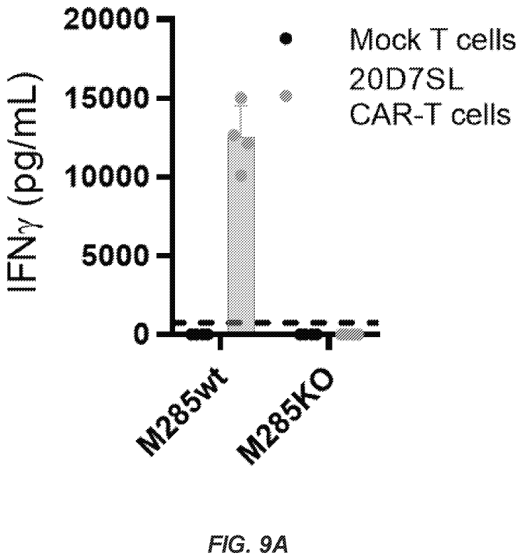

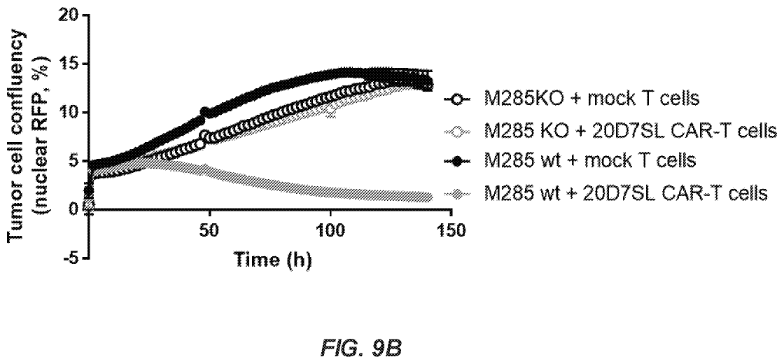

Human PBMCs were activated, transduced with lentiviral vectors encoding for the 20D7SL CAR construct with CD28 co-stimulatory signaling domain and expanded for 9 days. CAR-T cells and controls were co-cultured with M285 human melanoma cell line wild-type (with high expression of TYRP-1) or M285-TYRP-1 knockout cell line using a 5:1 T cell product-to-melanoma cell ratio. Untransduced T cells (mock) were used as control. Twenty-four hours after co-culture, the supernatants were collected and the IFNγ secretion was quantified by ELISA (FIG. 9A). Cytotoxicity was measured over time (FIG. 9B). These melanoma tumor cell lines stably express nuclear RFP (nRFP). To measure cytotoxicity, the percentage of nRFP was followed over time. The results are shown in FIGS. 9A-9B. Mean±SD are shown in the graph. Treatment with CAR-T cells stimulated IFNγ expression and inhibited tumor cell growth in TYPR-1-expressing cells.

### Example 10: In Vitro Antitumor Activity of the 20D7SL CAR-T Cells with Different Co-Stimulatory Signaling Domains

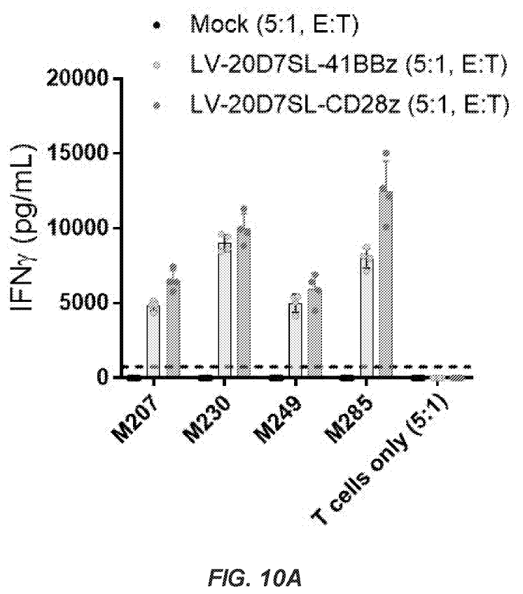

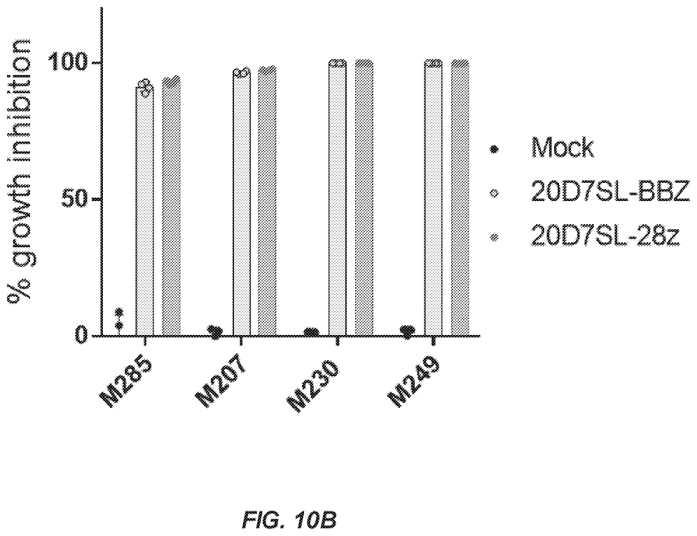

Human PBMCs were activated, transduced with lentiviral vectors encoding for the 20D7SL CAR construct with either 4-1BB costimulatory signaling domain (20D7SL-BBZ) or CD28 costimulatory signaling domain (20D7SL-28z) and expanded for 9 days. CAR-T cells and controls were co-cultured with a panel of melanoma cell lines with high expression of TYRP-1 using a 5:1 T cell product-to-melanoma cell ratio. Untransduced T cells (mock) were used as control. Twenty-four hours after co-culture, the supernatants were collected and the IFNγ secretion was quantified by ELISA (FIG. 10A). As an additional control, the secretion of interferon-gamma was also measured in the absence of target melanoma cells (RPMI, T cell only control). Percentage of growth inhibition was measured at 48h after co-culture (FIG. 10B). These melanoma tumor cell lines stably express nuclear RFP (nRFP). To measure cytotoxicity, the percentage of nRFP was followed over time. The percentage of nRFP in the tumor cell monolayers treated with CAR-T cells was normalized to the percentage of nRFP in the tumor cell monolayers treated with untransduced T cells to calculate the percentage of tumor growth inhibition. The results are shown in FIGS. 10A and 10B. Mean±SD and single values are shown in the graph. Both 20D7SL-BBZ and 20D7SL-28z stimulated IFNγ secretion and inhibited tumor cell growth.

### Example 11: In Vivo Antitumor Activity of the 20D7SL CAR T with Different Co-Stimulatory Signaling Domains in Patient-Derived Melanoma Models in Immunodeficient Mouse Models

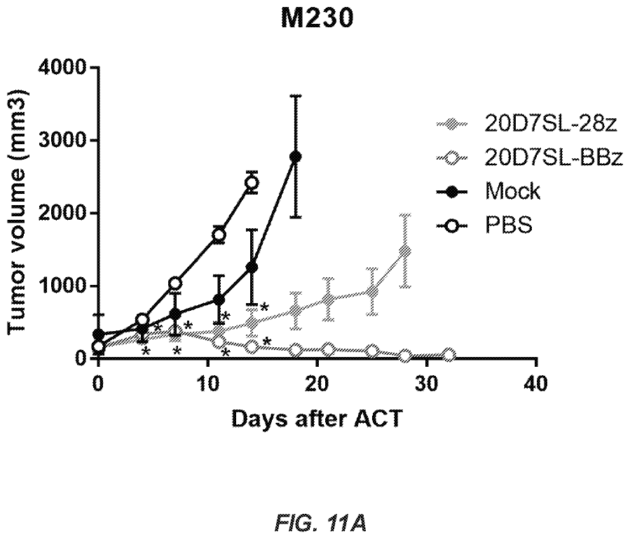

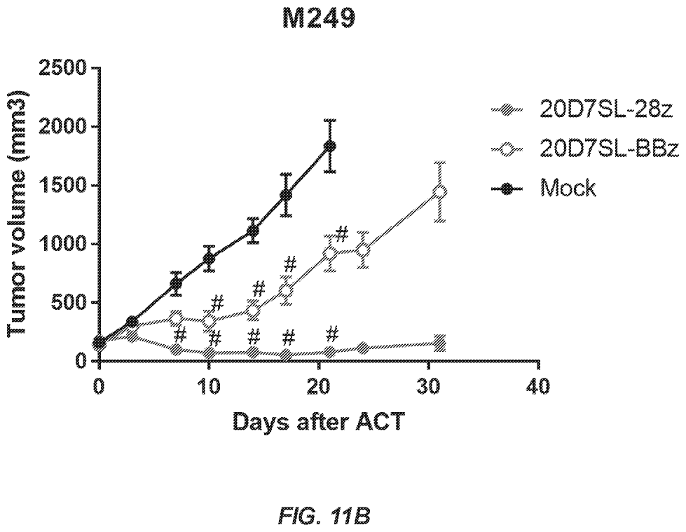

Human PBMCs were activated, transduced with lentiviral vectors encoding for the 20D7SL CAR construct with either 4-1BB costimulatory signaling domain (20D7SL-BBZ) or CD28 costimulatory signaling domain (20D7SL-28z) and expanded for 9 days. 10 million T cells transduced with the lentiviral vectors were administered intravenously into NSG mice bearing M230 (FIG. 11A) and M249 (FIG. 11B) subcutaneous tumors. The tumor volumes were followed overtime using a caliper (mean±SD are shown in the graph). Untransduced T cells or vehicle were used as a negative control. The results are shown in FIGS. 11A and 11B. * p<0.05 vs PBS; #p<0.05 vs untransduced T cells, unpaired t-test with Holm-Sidak correction for multiple comparison. Both 20D7SL-BBZ and 20D7SL-28z inhibited tumor growth.

### Example 12: TYRP-1 Expression in all Patients Combining TCGA Dataset, and BMS-CA029 and MK3475-001 Clinical Trial Datasets

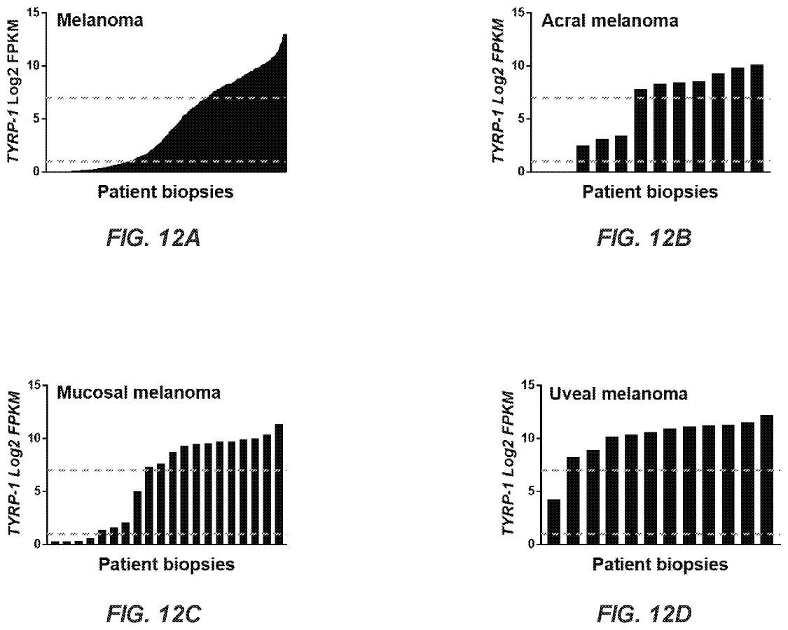

TYRP-1 expression data from various databases was analyzed. FIG. 12A shows TYRP-1 expression for all melanoma patients, FIG. 12B shows TYRP-1 expression for acral melanoma patients. FIG. 12C shows TYRP-1 expression for mucosal melanoma patients. FIG. 12D shows TYRP-1 expression for uveal melanoma patients. Grey dotted lines indicate positive TYRP-1 expression (≥1 Log 2 FPKM) and high TYRP expression (≥7 Log 2 FPKM).

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

The references recited in the application, to the extent that they provide exemplary procedural or other details supplementary to those set forth herein, are specifically incorporated herein by reference.

### REFERENCES

The following references and the publications referred to throughout the specification, to the extent that they provide exemplary procedural or other details supplementary to those set forth herein, are specifically incorporated herein by reference.
Patel D, Balderes P, Lahiji A, Melchior M, Ng S, Bassi R, et al. Generation and characterization of a therapeutic human antibody to melanoma antigen TYRP1. Hum Antibodies. 2007; 16(3-4):127-36.
Zhu E F, Gai S A, Opel C F, Kwan B H, Surana R, Mihm M C, et al. Synergistic innate and adaptive immune response to combination immunotherapy with anti-tumor antigen antibodies and extended serum half-life IL-2. Cancer Cell. 2015; 27(4):489-501.
Moynihan K D, Opel C F, Szeto G L, Tzeng A, Zhu E F, Engreitz J M, et al. Eradication of large established tumors in mice by combination immunotherapy that engages innate and adaptive immune responses. Nature medicine. 2016; 22(12):1402-10
Khalil D N, Postow M A, Ibrahim N, Ludwig D L, Cosaert J, Kambhampati S R, et al. An Open-Label, Dose-Escalation Phase I Study of Anti-TYRP1 Monoclonal Antibody IMC-20D7S for Patients with Relapsed or Refractory Melanoma. Clinical cancer research:an official journal of the American Association for Cancer Research. 2016; 22(21):5204-10.

1. A chimeric polypeptide comprising:
(a) an antigen binding domain comprising:
(i) a variable heavy (VH) region having at least 90% sequence identity with SEQ ID NO:10; and a variable light (VL) region having at least 90% sequence identity with SEQ ID NO:5; and/or
(ii) a variable heavy (VH) region comprising SEQ ID NO:11 (HCDR1), SEQ ID NO:12 (HCDR2), and SEQ ID NO:13 (HCDR3); and a variable light (VL) region comprising SEQ ID NO:6 (LCDR1), SEQ ID NO:7 (LCDR2), and SEQ ID NO:8 (LCDR3):
(b) a transmembrane domain; and
(c) an intracellular signaling domain.

2. (canceled)

3. The chimeric polypeptide of claim 1, wherein the VH region and the VL region are separated by a linker.

4. The chimeric polypeptide of claim 1, wherein the linker is between 4 and 40 amino acids in length.

5. The chimeric polypeptide of claim 4, wherein the linker comprises a Whitlow linker, (G₄S)ₙ, or (EAAAK)ₙ sequence, wherein n is 1, 2, 3, 4, 5, or 6.

6-7. (canceled)

8. The chimeric polypeptide of claim 1, further comprising a signal peptide.

9-10. (canceled)

11. The chimeric polypeptide of claim 1, wherein the transmembrane domain is an alpha or beta chain of the T cell receptor or a transmembrane domain from CD28, CD3ε (epsilon), CD45, CD4, CD5, CD8, CD9, CD16, CD22, CD33, CD37, CD64, CD80, CD86, CD123, CD134, CD137 or CD154.

12. The chimeric polypeptide of claim 11, wherein the transmembrane domain is a CD28 transmembrane domain.

13. (canceled)

14. The chimeric polypeptide of claim 1, wherein the intracellular signaling domain comprises a CD3ζ (zeta) signaling domain.

15-17. (canceled)

18. The chimeric polypeptide of claim 1, wherein the intracellular signaling domain further comprises a costimulatory domain, and wherein the costimulatory domain comprises a signaling domain from 4-1BB (CD137), CD28, IL-15Ra, OX40, CD2, CD27, CDS, ICAM-1, LFA-1 (CD11a/CD18), or ICOS (CD278).

19. The chimeric polypeptide of claim 18, wherein the costimulatory domain comprises a CD28 or 4-1BB signaling domain.

20-22. (canceled)

23. The chimeric polypeptide of claim 1, wherein the VH region is closer to the amino terminus of the chimeric polypeptide relative to the VL region.

24. The chimeric polypeptide of claim 1, wherein the VL region is closer to the amino terminus of the chimeric polypeptide relative to the VH region.

25. (canceled)

26. The chimeric polypeptide of claim 1, wherein the antigen binding domain and the transmembrane domain are separated by a hinge region, and wherein the hinge region comprises an IgG4 hinge, an IgG4-CH3 hinge, an IgG4-CH2CH3 hinge, a CD8α hinge, an IgG1 hinge, or a CD34 hinge.

27. The chimeric polypeptide of claim 26, wherein the hinge region is between 8 and 300 amino acids in length.

28-48. (canceled)

49. A nucleic acid comprising a sequence encoding the chimeric polypeptide of claim 1.

50-53. (canceled)

54. A cell comprising the nucleic acid of claim 49.

55-61. (canceled)

62. A population of cells comprising the cell of claim 54.

63. A pharmaceutical composition comprising (a) the population of cells of claim 62 and (b) a pharmaceutically acceptable excipient.

64. A method of making an engineered cell comprising introducing into a cell the nucleic acid of claim 49.

65-71. (canceled)

72. A method for treating a subject with cancer comprising administering to the subject (a) an effective amount of the population of cells of claim 62.

73-103. (canceled)
